(19) United States
(12) Patent Application Publication
(10) Pub. No.: US 2003/0082776 A1
(43) Pub. Date: May 1, 2003

(54) Novel genes encoding protein kinase/protein phosphatase

(76) Inventors: Toshio Ota, Tokyo (JP); Takao Isogai, Ibaraki (JP); Tetsuo Nishikawa, Tokyo (JP); Koji Hayashi, Osaka (JP); Kaoru Otsuka, Saitama (JP); Jun-Ichi Yamamoto, Chiba (JP); Shizuko Ishii, Chiba (JP); Tomoyasu Sugiyama, Tokyo (JP); Ai Wakamatsu, Chiba (JP); Keiichi Nagai, Tokyo (JP); Tetsuji Otsuki, Chiba (JP); Shin-Ichi Funahashi, Ibaraki (JP); Chiaki Senoo, Shizuoka (JP); Jun-Ichi Nezu, Ibaraki (JP)

Correspondence Address: JANIS K. FRASER, PH.D., J.D., FISH & RICHARDSON P.C., 225 Franklin Street, Boston, MA, 02110-2804, US

(21) Appl. No.: 10/059,585

(22) Filed: Jan. 29, 2002

Related U.S. Application Data
(63) Continuation-in-part of application No. PCT/JP00/05060, filed on Jul. 28, 2000.
(60) Provisional application No. 60/159,590, filed on Oct. 18, 1999.
(60) Provisional application No. 60/183,322, filed on Feb. 17, 2000.

(30) Foreign Application Priority Data
Jul. 29, 1999 (JP) 11-248036
Jan. 11, 2000 (JP) 2000-118776
May 2, 2000 (JP) 2000-183767

(51) Int. Cl. C12N 9/12; C12N 9/16; C07H 21/04; C12P 21/02; C12N 5/06

(52) U.S. Cl. 435/194; 435/196; 435/69.1; 435/320.1; 435/325; 536/23.2

(57) ABSTRACT

Selection of clones having the kinase and/or phosphatase-like structure from clones which had been isolated and the structures thereof had been determined in the Helix Research Institute (helix clones; Japanese Patent Application No. 2000-183767) was conducted. Twelve novel genes were provided by carrying out homology search for all the helix clones by using the amino acid sequences of known kinases and phosphatases as queries. The genes are expected to be involved in intracellular signal transduction. The physiological functions of the inventive genes can be tested by using reporter gene assay systems capable of detecting signal transduction. The proteins of the present invention are useful as target molecules in drug discovery and in the development of new pharmaceuticals.

This application is a continuation-in-part of PCT/JP00/05060, filed Jul. 28, 2000, which claims priority to U.S. Provisional Application Nos. 60/159,590, filed Oct. 18, 1999, and 60/183,322, filed Feb. 17, 2000; and Japanese Patent Application Nos. 11 -248036, filed Jul. 29, 1999; 2000-118776, filed Jan. 11, 2000; and 2000-183767, filed May 2, 2000.

### TECHNICAL FIELD

The present invention relates to novel human protein kinases and protein phosphatases, as well as to genes encoding the proteins.

### BACKGROUND

A variety of physiological functions of cells have to be regulated correctly and harmoniously according to need for cells to differtiate/proliferate into normal cells, and further to exert functions at the tissue level. It has been well known that the regulation of the state of protein phosphorylation by protein phosphorylation enzyme/protein kinase (hereinafter referred to as “kinase”) and protein dephosphorylation enzyme/protein phosphatase (hereinafter referred to as “phosphatase”) plays a central role in most of such regulatory mechanisms.

Many kinase and phosphatase genes have been identified to date. It has been clarified that they form a very large protein family with a well conserved structure (Semin. Cell Biol. 5(6):367-76, 1994; Cell 80(2):225-36, 1995; Genes Cells 1(2):147-69, 1996; Trends Biochem. Sci. 22(1):18-22, 1997; Proc Natl Acad Sci USA, 96(24):13603-10, 1999). The presence of numerous types of kinases and phosphatases in cells suggests that many types of intracellular physiological functions are precisely regulated by kinases and phosphatases. Thus, there is a possibility that agents acting on kinase or phosphatase can more precisely control physiological functions as compared with known agents represented by receptor agonist or receptor antagonist. Therefore, it is expected that agents acting on kinase or phosphatase are agents, which undesirable side effects can be much easily separated from the main effects, and accordingly, may function as highly useful pharmaceuticals.

In order to develop such agents acting on kinase or phosphatase, first, it is required to specify the intracellular physiological function associated with each of the kinases and phosphatases, and gain some information indicating the medical usefulness of suppressing or activating the function. Many types of kinases and phosphatases have been already isolated and studied. However, there may exist many unidentified molecules. Furthermore, with respect to kinases and phosphatases the genes of which have been isolated, it can be stated that information on intracellular physiological functions related with each kinase or phosphatase still are poor and has to be clarified. The identification of new kinase and phosphatase as well as clarification of physiological functions thereof is expected to make significant progress in the development of new pharmaceuticals and therapies.

### SUMMARY

The object of the present invention is to provide novel human protein kinase and protein phosphatase proteins, genes encoding the proteins, as well as production and uses of the same.

To accomplish the object described above, the present inventors strenuously carried out researches as follows. First, the present inventors tried to select clones having the kinase/phosphatase-like structure (KP clones) from clones which had been isolated and the structures of which had been determined in the Helix Research Institute (hereinafter referred to as “helix clones”; Japanese Patent Application No. Hei 11-248036). These helix clones are highly expected to have the full-length sequence, which were obtained by the combined use of; [1] preparation of a cDNA library containing sequences of full-length at a high rate achieved by the oligo-capping method; and [2] evaluation system for the completeness in cDNA length based on the 5′-end sequence (the selection is achieved based on the evaluation using ATGpr after eliminating non-fall length clones as compared with an EST). In addition, they are highly advantageous since the cDNAs are already inserted into a mammalian expression vector, they can be used promptly in experiments for the expression in cells.

The present inventors carried out homology search for all the helix clones using the amino acid sequences of known kinases and phosphatases as queries, and selected 12 clones: “C-NT2RP2000668”, “C-HEMBA1002212”, “C-NT2RM4001411 ”, “C-NT2RM4001758”, “C-NT2RP2002710”, “C-NT2RP2004933”, “C-PLACE1011923”, “C-NT2RP2001839”, “C-HEMBA1006173”, “C-OVARC 1000556”, “C-PLACE2000034”, and “C-HEMBA1001019” (hereinafter referred to as “KP clones”). These KP clones contain full-length cDNAs encoding novel human proteins. It has been known that many of known kinases and phosphatases are associated with a variety of signal transduction pathways in cells. Therefore, there is the possibility that the newly found KP clones having the kinase/phosphatase-like structure are also associated with some signal transduction pathways. The potential of the KP clones as target molecules in drug discovery can be explored through evaluating these KP clones in various assay systems using reporter genes and deducing the physiological functions thereof.

As described above, the present inventors found novel kinase/phosphatase proteins, and thereby accomplished the present invention.

Specifically, the present invention relates to novel human protein kinase and protein phosphatase proteins, genes encoding the proteins, and production and uses of the proteins and genes. More specifically, the present invention provides the following:

[1] a DNA of any one of the following (a) to (d):

(a) a DNA encoding a protein consisting of the amino acid sequence of SEQ ID NO:2, 4, 6, 8, 10, 12, 14, 16, 18, 20, or 22,

(b) a DNA comprising the coding region of the nucleotide sequence of SEQ ID NO: 1, 3, 5, 7, 9, 11, 13, 15, 17, 19, or 21,

(c) a DNA encoding a protein which (i) comprises the amino acid sequence of SEQ ID NO:2, 4, 6, 8, 10, 12, 14, 16, 18, 20, or 22 in which one or more amino acids are substituted, deleted, inserted and/or added, and (ii) is functionally equivalent to the protein consisting of the amino acid sequence of SEQ ID NO:2, 4, 6, 8, 10, 12, 14, 16, 18, 20, or 22, and

(d) a DNA hybridizing under a stringent condition to a DNA consisting of the nucleotide sequence of SEQ ID NO:1, 3, 5, 7, 9, 11, 13, 15, 17, 19, or 21, which encodes a protein functionally equivalent to the protein consisting of the amino acid sequence of SEQ ID NO:2, 4, 6, 8, 10, 12, 14, 16, 18, 20, or 22;

[2] a DNA encoding a partial peptide of a protein consisting of the amino acid sequence of SEQ ID NO:2, 4, 6, 8, 10, 12, 14, 16, 18, 20, or 22;

[3] a DNA comprising the nucleotide sequence of SEQ ID NO:24;

[4] a protein or partial peptide encoded by the DNA of [1] or [2];

[5] a polypeptide comprising the amino acid sequence of SEQ ID NO:25;

[6] a vector into which a DNA of any one of [1] to [3] has been inserted;

[7] a host cell containing a DNA of any one of [1] to [3], or the vector of [6];

[8] a method for producing the protein or peptide of [4] or [5], which comprises the steps of culturing the host cell of [7], and recovering the expressed protein from the host cell or the culture supernatant;

[9] an antibody binding to the protein or peptide of [4] or [5];

5[10] a polynucleotide containing at least 15 nucleotides complementary to a DNA consisting of the nucleotide sequence of SEQ ID NO:1, 3, 5, 7, 9, 11, 13, 15, 17, 19, or 21 or the complementary strand thereof; and

[11] a method of screening for a compound that binds to the protein or peptide of [4] or [5], which comprises the steps of:

(a) contacting a test sample containing at least one compound with the protein or peptide,

(b) detecting the binding activity of the compound with the protein or peptide, and

(c) selecting the compound that has the activity of binding to the protein or peptide.

The present invention provides human-derived genes “C-NT2RP2000668”, “C-HEMBA1002212”, “C-NT2RM4001411”, “C-NT2RM4001758”, “C-NT2RP2002710”, “C-NT2RP2004933”, “C-PLACE1011923”, “C-NT2RP2001839”, “C-HEMBA1006173”, “C-OVARC1000556”, “C-PLACE2000034”, and “C-HEMBA1001019” that encode novel kinases/phosphatases. SEQ ID NOs of the nucleotide sequences of these human-derived cDNAs and the amino acid sequences encoded by the cDNAs are shown below.

| Gene name | cDNA | Protein |
|---|---|---|
| C-NT2RP2000668 | SEQ ID NO:1 | SEQ ID NO:2 |
| C-HEMBA1002212 | SEQ ID NO:3 | SEQ ID NO:4 |
| C-NT2RM4001411 | SEQ ID NO:5 | SEQ ID NO:6 |
| C-NT2RM4001758 | SEQ ID NO:7 | SEQ ID NO:8 |
| C-NT2RP2002710 | SEQ ID NO:9 | SEQ ID NO:10 |
| C-NT2RP2004933 | SEQ ID NO:11 | SEQ ID NO:12 |
| C-PLACE1011923 | SEQ ID NO:13 | SEQ ID NO:14 |
| C-NT2RP2001839 | SEQ ID NO:15 | SEQ ID NO:16 |
| C-HEMBA1006173 | SEQ ID NO:17 | SEQ ID NO:18 |
| C-OVARC1000556 | SEQ ID NO:19 | SEQ ID NO:20 |
| C-PLACE2000034 | SEQ ID NO:21 | SEQ ID NO:22 |
| C-HEMBA1001019 | SEQ ID NO:23 | |

Further, the nucleotide sequence of a partial fragment of CDNA “C-HEMBA100 119” of SEQ ID NO:23 is shown in SEQ ID NO:24, and the amino acid sequence of the protein encoded by the cDNA fragment is shown in SEQ ID NO:25.

Hereinafter, unless otherwise stated, the above-mentioned genes of the present invention, “C-NT2RP2000668”, “C-HEMBA1002212”, “C-NT2RM4001411 ”, “C-NT2RM4001758”, “C-NT2RP2002710”, “C-NT2RP2004933”, “C-PLACE1011923”, “C-NT2RP2001839”, “C-HEMBA1006173”, “C-OVARC1000556”, “C-PLACE2000034”, and “C-HEMBA1001019” are collectively called “KP genes”, and proteins encoded by respective genes are collectively called “KP proteins” (“C-HEMBA1001019” is a protein consisting of the amino acid sequence of SEQ ID NO:25).

The inventive KP proteins were selected as clones having the kinase/phosphatase-like structure from the clones isolated and whose structures had been already determined in the Helix Research Institute. The regulation of the phosphorylation state of proteins by kinase and phosphatase plays central roles in normal differentiation and/or proliferation of cells, as well as in physiological functions at the cellular level. Thus, the inventive proteins are expected to share important functions in living body, and therefore, are useful as target molecules in drug development. In addition, the inventive KP proteins can be used as reagents for phosphorylating or dephosphorylating proteins.

The helix clones were prepared by a special method, and are expected to contain cDNA of full-length chains in high probability (Japanese Patent Application No. Hei 11-248036; Japanese Patent Application No. 2000-118776; Japanese Patent Application No. 2000-183767). Furthermore, because the cDNAs are already inserted in a mammalian expression vector, they can be used promptly in experiments for the expression in cells. Thus, information on physiological functions of the genes can be gained by successively testing these vectors with various assay systems using reporter genes. It has been known that many of known kinases and phosphatases are associated with a variety of signal transduction pathways in cells, and thus, the inventive KP genes can be also associated with signal transduction. Various potential physiological functions of the inventive genes can be thoroughly examined by functional screening using reporter gene assay systems in which known types of signal transduction can be detected.

Assay systems using reporter genes are excellent experimental systems which enable assessment of a variety of intracellular physiological functions simply in a single format. Specifically, the functional screening is preformed by the following reporter gene assay. A vector containing the inventive KP gene is introduced into the host cell with reporter genes having a variety of enhancer elements, and the KP gene is expressed in the cell. When the expression level of the reporter gene is altered as compared to that of the control cells in which no vector containing the KP gene had been introduced, it can be concluded that the protein encoded by the KP gene acted on the enhancer element. Useful information on physiological functions of the inventive KP gene is expected to be provided by testing whether the inventive KP gene acts on a variety of enhancer elements or not. Large amount of information on signal transduction systems acting on the elements, functional genes regulated by the enhancer elements, and so on, are known for many enhancer elements. Thus, when a KP gene being tested is proved to act on an enhancer element, it is possible to deduce physiological functions in which the KP gene participates based on known information on the enhancer element.

In the functional screening, it is also beneficial to study not only actions of a KP gene expressed alone, but also influences of the KP gene on the action after some stimuli. More specifically, even if the KP gene alone does not exhibit any activity, there is the possibility that the activation of a particular element by a known type of stimulus is enhanced or suppressed by the coexpressed KP gene. Such a known type of stimulus includes, for example, ligands of a cell surface receptor (interleukins, growth factors, TGF-P family, TNF-A family, hormones, low-molecular-weight compounds, etc.); expression of factors associated with intracellular signal transduction (various kinases, various phosphatases, low-molecular-weight G protein binding protein family, Smad family, STAT family, TRAF family, cell surface receptors, etc.); stress stimuli (oxidation stress, mechanical stress, heat stress, etc.); and so on.

The assays using reporter genes can be conducted by those skilled in the art by using a variety of commercially available kits that are used conventionally. For example, Mercury™ Pathway Profiling Systems from Clontech, PathDetectR Trans-Reporting System and PathDetectR Cis-Reporting System from Stratagene, and such are included. The assays can be conducted according to standard methods as described in the literature (“Overview of Genetic Reporter Systems” In Current Protocols in Molecular Biology, Ed. Ausubel, F. M. et al., (Wiley & Sons, N.Y.) Unit 9.6 (1995); Molecular Cloning: A Laboratory Manual, Cold Spring Harbor Laboratory Press (Cold Spring Harbor, N.Y. (1989)).

When the luciferase gene is used as the reporter gene, the luciferase activity can be measured, for example, by a standard method using Dual-Luciferase™ Reporter Assay System from Promega or the like.

Reporter genes that can be used in the above-mentioned functional screening include, for example, secretory alkaline phosphatase gene, chloramphenicol acetyltransferase (CAT) gene, β-galactosidase gene, and such in addition to luciferase gene. Further, enhancer elements that are used in the reporter assay can be exemplified by Serum Response Element (SRE), cAMP Response Element (CRE), TPA Response Element (TRE), NFKB (Nuclear factor of KB cell)-binding element, Heat shock Response Element (HRE), Glucocorticoid Response Element (GRE), AP 1 (Activator protein 1: c-jun/c-fos complex)-binding element, NFAT (Nuclear Factor of Activated T-cells)-binding element, p53-binding element, interferony activated element (Interferon Gamma Activated Sequence: GAS), Interferon-Stimulated Response Element (ISRE), E2F-binding element, STAT family-binding element, Smad family-binding element, TCF/LEF-binding element, GATA family-binding element, Sterol Regulatory Element (SRE), IRF (Interferon Regulatory Factor) family-binding element, PPAR Υ-binding element and AhR-binding element.

293 cell, Hela, NIH3T3, CV-1, Jurkat, vascular smooth muscle cell, vascular endothelial cell, and cardiac muscle cell can be exemplified as host cells that are used in the reporter assay.

Functionally equivalent proteins to the human KP proteins (SEQ ID NOs:2, 4, 6, 8, 10, 12, 14, 16, 18, 20, 22, and 25) are encompassed in the present invention. Such proteins include, for example, mutants, homologues, variants, and so on, of human KP proteins. The term “functionally equivalent” herein means that the protein of interest has a function of phosphorylating proteins and/or dephosphorylating proteins like the KP proteins. According to the following procedure, it can be judged whether or not the protein of interest phosphorylates a protein.

A kinase protein and a substrate protein are combined together in an appropriate reaction solution. After the reaction is conduced in the presence of ATP, the phosphorylation state of the substrate protein is measured to judge the phosphorylation activity. The kinase protein to be used can be purified from appropriate cell lines or extracts from tissue by commonly used biochemical methods. It is also possible to use kinase proteins obtained by the overexpression of introduced genes encoding kinase proteins into mammalian cells (COS7, CV-1, HEK293, HeLa, Jurkat, NIH3T3, etc.), insect cells (Sf9, etc.), E. coli, yeast, and so on. The phosphorylation state of the substrate protein can be measured in a liquid scintillation counter, autoradiography, and such, by using ATP labeled with radioisotope, such as [Υ-³²P] ATP.

Further, the phosphorylation state of the substrate protein can be measured by ELISA (enzyme-linked immunosorbent assay), Western blotting, etc. using phosphorylated protein specific antibodies or the like. Such substrate proteins to be used include proteins specific to particular kinases, as well as a variety of proteins, such as casein, histone, and myelin basic protein (MBP), which are known to be phosphorylated by non-specific kinases. Alternatively, synthetic peptides and such containing sequences that are phosphorylated may be also used.

Furthermore, the phosphorylation activity can be assessed by measuring the phosphorylation of the kinase proteinper se (autophosphorylation). More specifically, the assay can be performed according to conventional methods described in Protein Phosphorylation: A Practical Approach. First Edition (Hardie et al., Oxford University Press, 1993) or others.

It can be judged whether a protein of interest dephosphorylates a protein or not by using the following procedure.

A phosphatase protein and a pre-phosphorylated substrate protein are combined together in an appropriate reaction solution. Then, the decrease in the extent of phosphorylation of the substrate protein or the amount of phosphate released from the substrate protein is measured to assess the dephosphorylation activity. Those phosphatase proteins prepared by the same method as those described above for the assessment of the phosphorylation activity can be used as the phosphatase protein in this method. The same substrate protein mentioned above for the judgment of the phosphorylation activity can be used as the substrate protein herein. In addition, phosphorylase, phosphorylase kinase, and such can be also used as substrate proteins. The pre-phosphorylation of the substrate protein can be achieved by using appropriate kinase such as phosphorylase kinase, protein kinase A, tyrosine kinases including EGF receptor and so on. The phosphorylation state of the substrate protein can be assayed by the same method described above for the assessment of the phosphorylation activity. More specifically, the assay can be performed according to conventional methods described in “Protein Phosphorylation: A Practical Approach. First Edition (Hardie DG. et al., Oxford University Press, 1993)”, and so on.

Further, the substrate protein to be phosphorylated or dephosphorylated by a test protein can be identified by expressing a cDNA expression library composed of phage vectors or the like, and assessing whether a protein expressed from each clone can be a substrate for the test protein or not. More specifically, the identification can be carried out by referring to the method described in “EMBO J. (1997) 16:1921-1933”. Alternatively, the substrate protein can be identified through the identification of proteins binding to the test protein by the yeast two-hybrid screening or the like. More specifically, the identification can be carried out by referring to the method described in “EMBO J. (1997) 16:1909-1920”.

One method for preparing functionally equivalent proteins well known to those skilled in the art involves the introduction of mutations into the proteins. For example, one skilled in the art can prepare proteins functionally equivalent to the human KP protein (SEQ ID NO:2, 4, 6, 8, 10, 12, 14, 16, 18, 20, 22, or 25) by introducing appropriate mutations into the amino acid sequence of the protein using the site-directed mutagenesis method (Hashimoto-Gotoh et al., Gene 152:271-275, 1995; Zoller et al., Methods Enzymol. 100:468-500, 1983; Kramer et al., Nucleic Acids Res. 12:9441-9456, 1984; Kramer et al., Methods. Enzymol. 154:350-367, 1987; Kunkel, Proc. Natl. Acad. Sci. USA 82:488-492, 1986; Kunkel, Methods Enzymol. 85:2763-2766, 1988) and such. Mutation of amino acids may occur in nature, too. The proteins of the present invention include proteins comprising the amino acid sequence of human KP protein (SEQ ID NO:2, 4, 6, 8, 10, 12, 14, 16, 18, 20, 22, or 25) in which one or more amino acids are mutated, so long as the resulting mutant protein is functionally equivalent to the protein. In such a mutant protein, the number of the amino acids to be mutated is usually 50 residues or less, preferably 30 residues or less, and more preferably 10 residues or less (e.g., 5 residues or less).

The amino acid residue to be mutated is preferably mutated into a different amino acid that allows the properties of the amino acid side-chain to be conserved. Examples of properties of amino acid side chains include: hydrophobic amino acids (A, I, L, M, F, P, W, Y, V), hydrophilic amino acids (R, D, N, C, E, Q, G, H, K, S, T), and amino acids comprising the following side chains: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W) (The parenthetic letters indicate the one-letter codes of amino acids).

It is well known that a protein having deletion, addition, and/or substitution of one or more amino acid residues in the sequence of a protein can retain the original biological activity (Mark et al., Proc. Natl. Acad. Sci. USA 81:5662-5666, 1984; Zoller et al., Nucleic Acids Res. 10:6487-6500, 1982; Wang et al., Science 224:1431-1433; Dalbadie-McFarland et al. Proc. Natl. Acad. Sci. USA 79:6409-6413, 1982).

A protein having the amino acid sequence of human KP protein to which one or more amino acid residues have been added, is exemplified by a fusion protein containing the human KP protein. Fusion proteins, in which the human KP protein is fused to other peptides or proteins, are included in the present invention. Fusion proteins can be made using techniques well known to those skilled in the art, for example, by linking the DNA encoding the human KP protein (SEQ ID NO:2, 4, 6, 8, 10, 12, 14, 16, 18, 20, 22, or 25) in frame with the DNA encoding other peptides or proteins, followed by inserting the DNA into an expression vector and expressing it in a host. There is no restriction as to the peptides or proteins to be fused to the protein of the present invention.

For instance, known peptides which may be used for the fusion include the FLAG peptide (Hopp et al., BioTechnology 6:1204-1210, 1988), 6x His that is made up of six histidine residues, 10x His, influenza hemagglutinin (HA), human c-myc fragment, VSV-GP fragment, p18HIV fragment, T7-tag, HSV-tag, E-tag, SV40 T antigen fragment, lck tag, α-tubulin fragment, B-tag, and Protein C fragment. Also, glutathione-S-transferase (GST), influenza hemagglutinin (HA), the constant region of immunoglobulin, β-galactosidase, maltose binding protein (MBP), and the like may be used as a protein to be fused with the protein of this invention. Fusion proteins can be prepared by fusing the DNA encoding these peptides or proteins, which are commercially available, with the DNA encoding the protein of the invention, and expressing the fused DNA.

An alternative method for preparing functionally equivalent proteins known to those skilled in the art utilizes, for example, the hybridization technique (Sambrook et al., Molecular Cloning 2nd ed. 9.47-9.58, Cold Spring Harbor Lab. Press, 1989). Generally, one skilled in the art can isolate DNAs highly homologous to the whole or part of the DNA sequence encoding the human KP protein (SEQ ID NO: 1, 3, 5, 7, 9, 11, 13, 15, 17, 19, 21, or 23), and then isolate proteins functionally equivalent to the human KP protein based on those DNAs isolated. The present invention includes proteins that are (i) encoded by a DNA hybridizing to a DNA encoding the human KP protein and (ii) functionally equivalent to the human KP protein. Such proteins include, for example, homologues derived from human and other animals (for example, protein encoded by a DNA from mouse, rat, rabbit, cattle, etc.).

Those skilled in the art can properly select hybridization conditions to be used for the isolation of DNAs encoding proteins functionally equivalent to the human KP protein. Hybridization conditions include low stringent conditions. Low stringent conditions may be, for example, 42° C. in 2x SSC and 0.1% SDS, preferably 50° C. in 2x SSC and 0.1% SDS for washing after hybridization. More preferably, high stringent conditions such as 65° C. in 0.1x SSC and 0.1% SDS may be chosen. DNA with higher homology may be efficiently obtained at higher temperature under these conditions. However, several factors are thought to influence the stringency of hybridization, such as temperatures and salt concentrations, and one skilled in the art can suitably select these factors to accomplish a similar stringency. More guidelines for the hybridization condition are available in the art, for example, in a reference by Sambrook et al. (1989, Molecular Cloning, A Laboratory Manual, Cold Spring Harbor Press, N.Y.) and in unit 2.10 of the reference by Ausubel et al. (1995, Current Protocols in Molecular Biology, John Wiley & Sons, N.Y.).

Also, in lieu of hybridization, it is also possible to isolate functionally equivalent proteins by a gene amplification method, such as PCR, by synthesizing sequences based on the sequence information of the DNA encoding the human KP protein (SEQ ID NO: 1, 3, 5, 7, 9, 11, 13, 15, 17, 19, 21, or 23) and using them as primers.

The proteins functionally equivalent to the human KP proteins encoded by the DNA isolated by the hybridization or gene amplification techniques, usually are highly homologous to the human KP proteins (SEQ ID NO:2, 4, 6, 8, 10, 12, 14, 16, 18, 20, 22, or 25) at the amino acid sequence level. The proteins of the invention include proteins functionally equivalent to the human KP protein and are highly homologous to the amino acid sequence of SEQ ID NO:2, 4, 6, 8, 10, 12, 14, 16, 18, 20, 22, or 25. “Highly homologous” means typically 65% or higher, preferably 75% or higher, more preferably 85% or higher, and even more preferably 95% or higher identity at the amino acid level. Homology between proteins can be determined according to the algorithm described in the literature (Wilbur et al., Proc. Natl. Acad. Sci. USA 80:726-730, 1983).

The term “substantially pure” as used herein in reference to a given polypeptide means that the polypeptide is substantially free from other biological macromolecules. For example, the substantially pure polypeptide is at least 75%, 80, 85, 95, or 99% pure by dry weight. Purity can be measured by any appropriate standard method known in the art, for example, by column chromatography, polyacrylamide gel electrophoresis, or HPLC analysis.

Accordingly, the invention includes a polypeptide having a sequence shown as SEQ ID NO:2, 4, 6, 8, 10, 12, 14, 16, 18, 20, 22 or 25. The invention also includes a polypeptide, or fragment thereof, that differs from the corresponding sequence shown as SEQ ID NO:2, 4, 6, 8, 10, 12, 14, 16, 18, 20, 22 or 25. The differences are, preferably, differences or changes at a non-essential residue or a conservative substitution. In one embodiment, the polypeptide includes an amino acid sequence at least about 60% identical to a sequence shown as SEQ ID NO:2, 4, 6, 8, 10, 12, 14, 16, 18, 20, 22 or 25, or a fragment thereof. Preferably, the polypeptide is at least 65%, 70%, 75%, 80%, 85%, 90%, 95%, 98%, 99% or more identical to SEQ ID NO:2, 4, 6, 8, 10, 12, 14, 16, 18, 20, 22 or 25 and has at least one protein kinase or protein phosphatase activity. Preferred polypeptide fragments of the invention are at least 10%, preferably at least 20%, 30%, 40%, 50%, 60%, 70%, or more, of the length of the sequence shown as SEQ ID NO:2, 4, 6, 8, 10, 12, 14, 16, 18, 20, 22 or 25 and have at least one protein kinase or protein phosphatase activity described herein. Or alternatively, the fragment can be merely an immunogenic fragment.

The proteins of the present invention may have variations in the amino acid sequence, molecular weight, isoelectric point, presence or absence of sugar chains, or form, depending on the cell or host used to produce them or the purification method utilized as described below. Nevertheless, so long as the protein obtained has a function equivalent to the human KP protein, it is within the scope of the present invention. For example, when the inventive protein is expressed in prokaryotic cells, e.g., E. coli, a methionine residue is added at the N-terminus of the original protein. The present invention also includes such proteins.

The proteins of the present invention can be prepared as recombinant proteins or as naturally occurring proteins, using methods commonly known in the art. The recombinant protein can be, for example, prepared as follows. The DNA encoding the protein of this invention (e.g., DNA having the nucleotide sequence of SEQ ID NO:1, 3, 5, 7, 9, 11, 13, 15, 17, 19, 21, or 23) is inserted into an appropriate expression vector, and introduced into suitable host cells. Subsequently, the resulting transformants, the host cell inserted with the expression vector, are recovered, extracted and then purified by chromatography utilizing ion exchange, reverse phase, or gel filtration, or by affinity chromatography with a column in which the antibodies against the protein of the present invention are fixed, or by a combination of these columns.

Alternatively, the protein of the invention can be prepared by expressing the protein in host cells (e.g., animal cells or E. coli) as a fusion protein with glutathione S transferase protein, or as a recombinant protein with multiple histidine residues. The expressed protein can be purified using a glutathione column or nickel column. Subsequently, if necessary, regions of the fusion protein (apart from the desired protein) can be digested and removed with thrombin, factor Xa, etc.

The natural protein corresponding to the protein of the invention can be isolated by methods well known in the art, for example, by purifying tissue or cell extracts containing a protein of the invention with an affinity column to which the antibody that binds to the protein of the present invention described below is bound. The antibody may be a polyclonal antibody or monoclonal antibody.

The present invention also includes partial peptides of the proteins of the present invention. The partial peptides of the present invention comprise at least 7 or more amino acids, preferably 8 or more amino acids, more preferably 9 or more amino acids. The partial peptides can be used, for example, for generating antibodies against the protein of the present invention, screening of compounds binding to the protein of the present invention, or screening of promoters or inhibitors for the protein of the present invention. The partial peptides can be used as antagonists or competitive inhibitors for the protein of this invention. The partial peptides of the invention can be produced by genetic engineering, known methods of peptide synthesis, or by digesting the protein of the invention with an appropriate peptidase. For peptide synthesis, for example, solid phase synthesis or liquid phase synthesis may be used.

DNA encoding an inventive protein can be used for the production of the inventive protein in vivo and in vitro as described above; it is also applicable to, for example, gene therapy for diseases caused by the abnormality in the gene encoding the inventive protein and for diseases that can be treated by the inventive protein. Any type of DNA, such as cDNA synthesized from MRNA, genomic DNA or synthetic DNA, can be used so long as the DNA encodes a protein of the present invention. Also so long as they can encode a protein of the present invention, DNAs comprising arbitrary sequences based on the degeneracy of the genetic code are also included.

The DNA of the present invention can be prepared using methods known in the art. For example, a cDNA library can be constructed from the cells expressing the protein of the present invention, and hybridization can be conducted using a part of the DNA sequence of the present invention (for example, SEQ ID NO: 1, 3, 5, 7, 9, 11, 13, 15, 17, 19, 21, or 23) as a probe. cDNA libraries may be prepared by, for example, the method described in the literature (Sambrook et al., Molecular Cloning, Cold Spring Harbor Laboratory Press, 1989), and also, commercially available ones can be used. Alternatively, the DNA of the present invention can be obtained by preparing the RNA from the cells expressing the protein of the present invention, synthesizing cDNA by reverse transcriptase, synthesizing the oligo-DNAs based on the DNA sequence of the present invention (for example, SEQ ID NO:1, 3, 5, 7, 9, 11, 13, 15, 17, 19, 21, or 23), and amplifying the cDNA encoding the protein of the present invention by PCR using the oligonucleotides as primers.

The nucleotide sequence of the obtained cDNA is determined to find an open reading frame, and thereby the amino acid sequence of the protein of the invention can be obtained. The cDNA obtained may also be used as a probe for screening a genomic library to isolate a genomic DNA.

More specifically, mRNAs may first be prepared from a cell, tissue, or organ in which the protein of the invention is expressed. Known methods can be used to isolate mRNAs; for instance, total RNA can be prepared by guanidine ultracentrifugation (Chirgwin et al., Biochemistry 18:5294-5299, 1979) or the AGPC method (Chomczynski et al., Anal. Biochem. 162:156-159, 1987). mRNA may then be purified from total RNA using mRNA Purification Kit (Pharmacia) and such; alternatively, mRNA may be directly purified by QuickPrep mRNA Purification Kit (Pharmacia).

The obtained mRNA is used to synthesize cDNA using reverse transcriptase. cDNA may be synthesized by using a kit such as the AMV Reverse Transcriptase First-strand cDNA Synthesis Kit (Seikagaku Kogyo). Alternatively, cDNA may be synthesized and amplified following the 5′-RACE method (Frohman et al., Proc. Natl. Acad. Sci. USA 85:8998-9002, 1988; Belyavsky et al., Nucleic Acids Res. 17:2919-2932, 1989) which uses primers described herein, the 5′-Ampli FINDER RACE Kit (Clontech), and polymerase chain reaction (PCR).

A desired DNA fragment is prepared from the PCR products and ligated with a vector DNA. The recombinant vectors are used to transform E. coli and such, and a desired recombinant vector is prepared from a selected colony. The nucleotide sequence of the desired DNA is verified by conventional methods, such as dideoxynucleotide chain termination.

A DNA of the invention may be designed to have a sequence that is expressed more efficiently by taking into account the frequency of codon usage in the host to be used for expression (Grantham et al., Nucleic Acids Res. 9:43-74, 1981). The DNA of the present invention may be altered by a commercially available kit or a conventional method. For instance, the DNA may be altered by digestion with restriction enzymes, insertion of a synthetic oligonucleotide or an appropriate DNA fragment, addition of a linker, or insertion of the initiation codon (ATG) and/or the stop codon (TAA, TGA, or TAG).

The DNAs of the present invention specifically include the DNA which comprises a region containing the following nucleotide sequence.

nucleotides from A at nucleotide residue 109 to T at nucleotide residue 1713 in the nucleotide sequence of SEQ ID NO: 1.

nucleotides from A at nucleotide residue 170 to C at nucleotide residue 1135 in the nucleotide sequence of SEQ ID NO:3.

nucleotides from A at nucleotide residue 173 to A at nucleotide residue 1450 in the nucleotide sequence of SEQ ID NO:5.

nucleotides from A at nucleotide residue 3 to A at nucleotide residue 1916 in the nucleotide sequence of SEQ ID NO:7.

nucleotides from A at nucleotide residue 71 to G at nucleotide residue 2479 in the nucleotide sequence of SEQ ID NO:9.

nucleotides from A at nucleotide residue 215 to C at nucleotide residue 1576 in the nucleotide sequence of SEQ ID NO: 11.

nucleotides from A at nucleotide residue 773 to C at nucleotide residue 2179 in the nucleotide sequence of SEQ ID NO: 13.

nucleotides from A at nucleotide residue 23 to G at nucleotide residue 2290 in the nucleotide sequence of SEQ ID NO:15.

nucleotides from A at nucleotide residue 67 to G at nucleotide residue 690 in the nucleotide sequence of SEQ ID NO:17.

nucleotides from A at nucleotide residue 1357 to A at nucleotide residue 1929 in the nucleotide sequence of SEQ ID NO: 19.

nucleotides from A at nucleotide residue 40 to C at nucleotide residue 2415 in the nucleotide sequence of SEQ ID NO:21.

nucleotides from A at nucleotide residue 1371 to A at nucleotide residue 1494 in the nucleotide sequence of SEQ ID NO:23.

The DNA of the present invention also include a DNA hybridizing to a DNA consisting of the nucleotide sequence of SEQ ID NO: 1, 3, 5, 7, 9, 11, 13, 15, 17, 19, 21, or 23 and encoding a protein functionally equivalent to the above-mentioned protein of the present invention. Those skilled in the art can properly select the appropriate hybridization conditions, and specifically the above-mentioned conditions can be used. Under these conditions, the higher the temperature, the higher the homology of the obtained DNA will be. The above-mentioned hybridizing DNA is preferably a naturally occurring DNA, for example, cDNA or chromosomal DNA.

As used herein, an “isolated nucleic acid” is a nucleic acid, the structure of which is not identical to that of any naturally occurring nucleic acid or to that of any fragment of a naturally occurring genomic nucleic acid spanning more than three genes. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in random, uncharacterized mixtures of different DNA molecules, transfected cells, or cell clones, e.g., as these occur in a DNA library such as a cDNA or genomic DNA library.

Accordingly, in one aspect, the invention provides an isolated or purified nucleic acid molecule that encodes a polypeptide described herein or a fragment thereof. Preferably, the isolated nucleic acid molecule includes a nucleotide sequence that is at least 60% identical to the nucleotide sequence shown in SEQ ID NO: 1, 3, 5, 7, 9, 11, 13, 15, 17, 19, 21, 23 or 24. More preferably, the isolated nucleic acid molecule is at least 65%, 70%, 75%, 80%, 85%, 90%, 91%, 92%, 93%, 94%, 95%, 96%, 97%, 98%, 99%, or more, identical to the nucleotide sequence shown in SEQ ID NO:1, 3, 5, 7, 9, 11, 13, 15, 17, 19, 21, 23 or 24. In the case of an isolated nucleic acid molecule which is longer than or equivalent in length to the reference sequence, e.g., SEQ ID NO:1, 3, 5, 7, 9, 11, 13, 15, 17, 19, 21, 23 or 24, the comparison is made with the full length of the reference sequence. Where the isolated nucleic acid molecule is shorter that the reference sequence, e.g., shorter than SEQ ID NO: 1, 3, 5, 7, 9, 11, 13, 15, 17, 19, 21, 23 or 24, the comparison is made to a segment of the reference sequence of the same length (excluding any loop required by the homology calculation).

As used herein, “% identity” of two amino acid sequences, or of two nucleic acid sequences, is determined using the algorithm of Karlin and Altschul (PNAS USA 87:2264-2268, 1990), modified as in Karlin and Altschul, PNAS USA 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score =100, wordlength =12. BLAST protein searches are performed with the XBLAST program, score =50, wordlength =3. To obtain gapped alignment for comparison purposes GappedBLAST is utilized as described in Altschul et al. (Nucleic Acids Res. 25:3389-3402, 1997). When utilizing BLAST and GappedBLAST programs the default parameters of the respective programs (e.g., XBLAST and NBLAST) are used to obtain nucleotide sequences homologous to a nucleic acid molecule of the invention.

The present invention also provides a vector into which a DNA of the present invention is inserted. The vectors of the present invention are useful for maintaining the DNA of the present invention within host cells or expressing the protein of the invention.

When the E. coli is used as a host cell, there is no limitation other than that the vector should have an “ori” to amplify and mass-produce the vector in E. Coli (e.g., JM109, DH5α, HB101, or XL1Blue), and a marker gene for selecting the transformed E. coli (e.g., a drug-resistance gene selected by a drug such as ampicillin, tetracycline, kanamycin, or chloramphenicol). For example, M13-series vectors, pUC-series vectors, pBR322, pBluescript, pCR-Script, and such can be used. pGEM-T, pDIRECT, pT7, and so on can also be used for subcloning and excision of the cDNA as well as the vectors described above. When a vector is used to produce a protein of the present invention, an expression vector is especially useful. The expression vector, for example, to be expressed in E. coli should have the above characteristics to be amplified in E. coli. When E. coli, such as JM109, DH5α, HB101, or XL1 Blue, is used as the host cell, the vector should have a promoter such as lacZ promoter (Ward et al., Nature 341:544-546, 1989; FASEB J. 6:2422-2427, 1992), araB promoter (Better et al., Science 240:1041-1043, 1988), or T7 promoter that can efficiently promote the expression of the desired gene in E. coli. Other examples of the vectors are pGEX-5X-1 (Pharmacia), “QlAexpress system” (Qiagen), pEGFP, and pET (for this vector, BL2 1, a strain expressing T7 RNA polymerase, is preferably used as the host).

Further, the vector may contain a signal sequence for the secretion of polypeptides. The pelB signal sequence (Lei, S. P. et al., J. Bacteriol. (1987) 169, 4379) can be used as a signal sequence for secretion of proteins, when the proteins are intended to be produced in the periplasm of E. coli. Introduction of the vector into a host cell can be performed, for example, by the calcium chloride method or electroporation.

In addition to the vectors for E. coli, for example, the vector for producing the proteins of this invention may be a mammal-derived expression vector (e.g., pcDNA3 (Invitrogen), pEGF-BOS (Nucleic Acids Res. 1990, 18(17), p5322), pEF, and pCDM8), an insect cell-derived expression vector (e.g., “Bac-to-BAC baculovairus expression system” (GibcoBRL) and pBacPAK8), a plant-derived expression vector (e.g., pMH1 and pMH2), an animal virus-derived expression vector (e.g., pHSV, pMV, and pAdexLcw), a retrovirus-derived expression vector (e.g., pZlPneo), an yeast-derived expression vector (e.g., “Pichia Expression Kit” (Invitrogen), pNV11, and SP-QO1), a Bacillus subtilis-derived expression vector (e.g., pPL608 and pKTH50).

In order to express proteins in animal cells, such as CHO, COS, and NIH3T3 cells, the vector should have a promoter necessary for expression in such cells, e.g., SV40 promoter (Mulligan et al., Nature 277:108, 1979), MMLV-LTR promoter, EFl α promoter (Mizushima et al., Nucleic Acids Res. 18:5322, 1990), CMV promoter, etc., and more preferably it has a marker gene for selecting transformants (for example, a drug resistance gene selected by a drug (e.g., neomycin, G418, etc.)). Examples of vectors with these characteristics include pMAM, pDR2, pBK-RSV, pBK-CMV, pOPRSV, pOP13, and so on.

The method using CHO cells deficient in nucleic acid synthetic pathways as the host, and incorporating a vector (such as pCHOI) with a DHFR gene that compensates for the deficiency and amplifying the vector with methotrexate (MTX) can be mentioned as an example method for stably expressing a gene and amplifying the copy number in cells. And as a method for transient expression, a method transforming the COS cells, which have the gene for SV40 T antigen on the chromosome, with a vector (such as pcD) having the SV40 replication origin can be mentioned. The origin used for replication may be those of polyomavirus, adenovirus, bovine papilloma virus (BPV), and the like. In addition, the expression vector may include a selection marker gene for amplification of the gene copies in host cells. Examples of such markers include, but are not limited to, the aminoglycoside transferase (APH) gene, the thymidine kinase (TK) gene, the E. coli xanthine-guanine phosphoribosyl transferase (Ecogpt) gene, and the dihydrofolate reductase (dhfr) gene.

The DNA of the present invention can be expressed in animals by, for example, inserting a DNA of the invention into an appropriate vector and introducing the vector into a living body by the retrovirus method, liposome method, cationic liposome method, adenovirus method, and so on. Thus, gene therapy can be conducted for diseases caused by mutations in the KP gene of this invention. The vectors used include, but are not limited to, adenoviral vectors (e.g., pAdexlcw) and retroviral vectors (e.g., pZlPneo). General techniques for gene manipulation, such as insertion of the DNA of the invention into a vector, can be performed according to conventional methods (Molecular Cloning, 5.61-5.63). The DNA of this invention can be administered to the living body by an ex vivo method or in vivo method.

The present invention also provides a host cell into which the vector of the present invention has been introduced. The host cell into which the vector of the invention is introduced is not particularly limited. E. coli and various animal cells can be used. The host cell of this invention can be used as, for example, a production system for producing or expressing the protein of the invention. The production system for producing a protein of the invention may be both in vitro or in vivo production system. For in vitro production, eukaryotic cells or prokaryotic cells can be used.

Useful eukaryotic host cells may be animal, plant, or fungi cells. As animal cells, mammalian cells such as CHO (J. Exp. Med. 108:945, 1995), COS, 3T3, myeloma, baby hamster kidney (BHK), HeLa, or Vero cells, amphibian cells such as Xenopus oocytes (Valle et al. Nature 291:340-358, 1981), or insect cells such as Sf9, Sf21, or Tn5 cells can be used. CHO cells lacking DHFR gene (dhfr-CHO) (Proc. Natl. Acad. Sci. USA 77:4216-4220, 1980) or CHO K-1 (Proc. Natl. Acad. Sci. USA 60:1275, 1968) may also be used. Among the animal cells, CHO cells are particularly preferable for high-level expression. The vector can be introduced into the host cell by, for example, the calcium phosphate method, the DEAE-dextran method, cationic liposome DOTAP (Boehringer Mannheim) method, electroporation, lipofection, etc.

As plant cells, for example, plant cells originating from Nicotiana tabacum are known as protein production system and may be used as callus cultures. As fungi cells, yeast cells such as Saccharomyces, including Saccharomyces cerevisiae, or filamentous fungi such as Aspergillus, including Aspergillus niger, are known.

Useful prokaryotic cells include bacterial cells, such as E. coli, for example, JM 109, DH5α, and HB101, or Bacillus subtilis.

These cells are transformed by a desired DNA, and the resulting transformants are cultured in vitro to obtain the protein. Transformants can be cultured using known methods. Culture medium such as DMEM, MEM, RPMI1640, or IMDM may be used for animal cells. The culture medium can be used with or without serum supplement such as fetal calf serum (FCS). The pH of the culture medium is preferably between about 6 and 8. Cells are typically cultured at about 30 to 40° C. for about 15 to 200 hr, and the culture medium may be replaced, aerated, or stirred if necessary.

Animal and plant hosts may be used for in vivo production. For example, a desired DNA can be introduced into an animal or plant host. Encoded proteins are produced in vivo, and then are recovered. These animal and plant hosts are included in host cells of the present invention.

Animals to be used for the production system described above include mammals and insects. Mammals such as goat, porcine, sheep, mouse, and bovine may be used (Vicki Glaser, SPECTRUM Biotechnology Applications (1993)). Alternatively, the mammals may be transgenic animals.

For instance, a desired DNA may be prepared as a fusion gene, fused with a gene such as goat β casein gene which encodes a protein specifically produced into milk. DNA fragments comprising the fusion gene are injected into goat embryos, which are then transplanted back to female goats. Proteins of interest can be recovered from milk produced by the transgenic goats (i.e., those born from the goats that had received the embryos) or from their offspring. To increase the amount of milk containing the proteins produced by transgenic goats, hormones may be appropriately administered to them (Ebert et al., Bio/Technology 12:699-702, 1994).

Alternatively, insects, such as the silkworm, may be used. Baculoviruses into which the DNA encoding the protein of interest is inserted can be used to infect silkworms, and the desired protein can be recovered from their body fluid (Susumu et al., Nature 315:592-594, 1985).

As plants, for example, tobacco can be used. In use of tobacco, DNA encoding the protein of interest may be inserted into a plant expression vector, such as pMON530, which is introduced into bacteria, such as Agrobacterium tumefaciens. Then the bacteria is used to infect tobacco, such as Nicotiana tabacum, and a desired polypeptide can be recovered from their leaves (Julian et al., Eur. J. Immunol. 24:131-138, 1994).

A protein of the present invention obtained as above may be isolated from inside or outside of the host cells (e.g., culture media), and purified as a substantially pure homogeneous protein. The method for protein isolation and purification is not limited to any specific method; in fact, any standard method may be used. For instance, column chromatography, filter, ultrafiltration, salt precipitation, solvent precipitation, solvent extraction, distillation, immunoprecipitation, SDS-polyacrylamide gel electrophoresis, isoelectric point electrophoresis, dialysis, recrystallization, and so on may be appropriately selected and combined to isolate and purify the protein.

For example, affinity chromatography, ion-exchange chromatography, hydrophobic chromatography, gel filtration, reverse phase chromatography, adsorption chromatography, and such may be used for chromatography (Strategies for Protein Purification and Characterization: A Laboratory Course Manual. Ed. Daniel R. Marshak et al., Cold Spring Harbor Laboratory Press (1996)). These chromatographies may be performed by liquid chromatography such as HPLC and FPLC. Thus, the present invention includes highly purified proteins, purified by the above methods.

A protein of the present invention may be optionally modified or partially deleted by treating it with an appropriate protein modification enzyme before or after purification. Useful protein modification enzymes include, but are not limited to, trypsin, chymotrypsin, lysylendopeptidase, protein kinase, glucosidase, and so on.

The present invention also provides antibodies that bind to the protein of the invention. The antibody of the invention may take any form, including monoclonal antibody, as well as polyclonal antibodies. Furthermore, antiserum obtained by immunizing an animal such as rabbit with the protein of the invention, all classes of polyclonal and monoclonal antibodies, human antibodies, and humanized antibodies produced by genetic recombination are included.

A protein of the invention used as the antigen to obtain antibodies may be derived from any animal species, but preferably it is derived from a mammal, such as a human, mouse, or rat, and more preferably from human. A human-derived protein may be obtained from the nucleotide or amino acid sequences disclosed herein.

Herein, a protein used as an antigen may be a complete protein or partial peptides thereof. A partial peptide may be, for example, an amino (N)-terminal or carboxy (C)-terminal fragment of the protein. Herein, an antibody is defined as an antibody that reacts with either the full-length or a fragment of the protein.

A gene encoding a protein of the invention or its fragment may be inserted into a known expression vector, which is used to transform a host cell as described herein. The desired protein or its fragment may be recovered from the outside or inside of the host cell by any standard method, and may be used as an antigen. Alternatively, cells expressing the protein or their lysates, or a chemically synthesized protein may be used as an antigen. Short peptides are preferably used as antigens by appropriately combining them with carrier proteins such as keyhole limpet hemocyanin, bovine serum albumin, and ovalbumin.

Any mammalian animal may be immunized with the antigen, but preferably the compatibility with parental cells used for cell fusion is taken into account. In general, animals of Rodentia, Lagomorpha, or Primates are used.

Animals of Rodentia include, for example, mouse, rat, and hamster. Animals of Lagomorpha include, for example, rabbit. Animals of Primates include, for example, a monkey of Catarrhini (old world monkey) such as crab-eating monkey, rhesus monkey, sacred baboon, or chimpanzee.

Methods for immunizing animals with antigens are known in the art. For instance, intraperitoneal injection or subcutaneous injection of antigens is used as a standard method for immunization of mammals. More specifically, antigens may be diluted and suspended in an appropriate amount with phosphate buffered saline (PBS), physiological saline, etc. If desired, the antigen suspension may be mixed with an appropriate amount of a standard adjuvant, such as Freund's complete adjuvant, made into emulsion, and then administered to mammals. Preferably, it is followed by several administrations of antigen mixed with an appropriately amount of Freund's incomplete adjuvant every 4 to 21 days. An appropriate carrier may also be used for immunization. After immunization as above, serum is examined for increase of the amount of desired antibodies by a standard method.

Polyclonal antibodies against the proteins of the present invention may be prepared by collecting blood from the immunized mammal examined for the increase of desired antibodies in the serum, and by separating serum from the blood by any conventional method. Serum containing the polyclonal antibodies, or if necessary, a fraction containing the polyclonal antibodies may be isolated from the serum to be used as the polyclonal antibodies of the present invention. For example, immunoglobulin G or M can be prepared by using an affinity column coupled with the protein of the invention to obtain the fraction exclusively recognizing the protein of the invention, and then, purifying the fraction by using protein A or protein G column.

To prepare monoclonal antibodies, immune cells are collected from the mammal immunized with the antigen and checked for the increased level of desired antibodies in the serum as described above, and are subjected to cell fusion. The immune cells used for cell fusion are preferably obtained from spleen. The other parent cell which is fused with the above immune cell is preferably a mammalian myeloma cell, and more preferably a myeloma cell that has acquired a special feature that can be used for selection of fusion cells by drugs.

Cell fusion of the above immune cell and myeloma cell may be performed by any standard method, such as those described in the literature (Galfre et al., Methods Enzymol. 73:3-46, 1981).

Hybridomas obtained by the cell fusion may be selected by cultivating them in a standard selection medium, such as HAT medium (hypoxanthine, aminopterin, and thymidine containing medium). The cell culture is typically continued in the HAT medium for several days to several weeks, the time being sufficient to allow all the other cells, except desired hybridoma (non-fused cells), to die. Then, the standard limiting dilution is performed to screen and clone a hybridoma cell producing the desired antibody.

Besides the above method, in which a nonhuman animal is immunized with an antigen for preparing hybridoma, human lymphocytes such as that infected by EB virus may be immunized with a protein, protein expressing cells, or their lysates in vitro. Then, the immunized lymphocytes are fused with human-derived myeloma cells that is capable of indefinitely dividing, such as U266, to yield a hybridoma producing a desired human antibody, able to bind to the protein can be obtained (Unexamined Published Japanese Patent Application (JP-A) No. Sho 63-17688).

Subsequently, the hybridomas thus obtained are transplanted into the abdominal cavity of a mouse from which the ascites is collected. The monoclonal antibodies thus obtained can be purified by, for example, ammonium sulfate precipitation or by column chromatography using a protein A or protein G column, a DEAE ion exchange column, an affinity column to which the protein of the invention is coupled, and such. The antibody of the invention can be used not only for purifying and detecting the protein of the invention, but also as a candidate for an agonist or antagonist to the protein of the present invention. It is also expected to use the antibody for antibody therapy of diseases associated with the protein of this invention. When the antibody obtained is administered to the human body (antibody therapy), human antibodies or humanized antibodies are preferred to reduce immunogenicity.

For example, transgenic animals having a repertory of human antibody genes may be immunized with a protein, protein expressing cells, or their lysates as an antigen. Antibody producing cells are collected from the animals, and fused with myeloma cells to obtain hybridoma, from which human antibodies against the protein can be prepared (see W092-03918, W093-2227, W094-02602, W094-25585, W096-33735, and W096-34096).

Alternatively, an immune cell, such as an immunized lymphocyte, producing antibodies may be immortalized by an oncogene and used for preparing monoclonal antibodies.

Monoclonal antibodies thus obtained can also be recombinantly prepared using genetic engineering techniques (see, for example, Borrebaeck C. A. K. and Larrick J. W. Therapeutic Monoclonal Antibodies, published in the United Kingdom by MacMillan Publishers LTD (1990)). A DNA encoding an antibody may be cloned from an immune cell, such as hybridomas or immunized lymphocytes producing the antibody; inserted into an appropriate vector; and introduced into host cells to prepare a recombinant antibody. The present invention also includes recombinant antibodies prepared as described above.

The antibody of the present invention may be a fragment of an antibody or modified antibody, so long as it binds to the protein of the invention. For instance, the antibody fragment may be Fab, F(ab′)₂, Fv, or single chain Fv (scFv), in which Fv fragments from H and L chains are ligated by an appropriate linker (Huston et al., Proc. Natl. Acad. Sci. USA 85:5879-5883, 1988). More specifically, an antibody fragment may be generated by treating an antibody with an enzyme such as papain or pepsin. Alternatively, a gene encoding the antibody fragment may be constructed; inserted into an expression vector; and expressed in an appropriate host cell (see, for example, Co et al., J. Immunol. 152:2968-2976, 1994; Better et al., Methods Enzymol. 178:476-496, 1989; Pluckthun et al., Methods Enzymol. 178:497-515, 1989; Lamoyi, Methods Enzymol. 121:652-663, 1986; Rousseaux et al., Methods Enzymol. 121:663-669, 1986; Bird et al., Trends Biotechnol. 9:132-137, 1991).

An antibody may be modified by conjugation with a variety of molecules, such as polyethylene glycol (PEG). The antibody of the present invention includes such modified antibodies. A modified antibody can be obtained by chemically modifying an antibody. These modification methods have been already established in the field.

Alternatively, the antibody of the present invention may be obtained as a chimeric antibody, between a variable region derived from nonhuman antibody and the constant region derived from human antibody, or as a humanized antibody, comprising the complementarity determining region (CDR) derived from nonhuman antibody, the frame work region (FR) derived from human antibody, and the constant region. Such antibodies can be prepared by using known technology.

Obtained antibodies may be purified to homogeneity. The antibodies can be separated and purified by using standard methods for protein separation and purification. For instance, column chromatography such as affinity chromatography, filter, ultrafiltration, salt precipitation, dialysis, SDS-polyacrylamide gel electrophoresis, isoelectric point electrophoresis, and so on may be appropriately selected and combined to isolate and purify the antibody (Antibodies: A Laboratory Manual. Ed Harlow and David Lane, Cold Spring Harbor Laboratory, 1988), but methods are not limited to them. The concentration of the antibody obtained as described above can be determined by the measurement of absorbance, enzyme-linked immunosorbent assay (ELISA), or others.

Columns for affinity chromatography include protein A column and protein G column. For example, protein A column includes Hyper D, POROS, Sepharose F. F. (Pharmacia) and the like.

In addition to affinity chromatography, chromatographic methods include, for example, ion exchange chromatography, hydrophobic chromatography, gel filtration, reverse-phase chromatography, adsorption chromatography and others (“Strategies for Protein Purification and Characterization: A Laboratory Course Manual” Ed Daniel R. Marshak et al., Cold Spring Harbor Laboratory Press, 1996). These chromatographic methods can be conducted by using liquid chromatography such as HPLC and FPLC.

For example, absorbance measurement, enzyme-linked immunosorbent assay (ELISA), enzyme immunoassay (EIA), radioimmunoassay (RIA), or immunofluorescence may be used to measure the antigen binding activity of the antibody of the invention. In ELISA, the antibody of the present invention is immobilized on a plate; the protein of the invention is applied to the plate; and then a sample containing a desired antibody, such as culture supernatant of antibody producing cells or purified antibodies, is applied. Then, a secondary antibody that recognizes the primary antibody and which is labeled with an enzyme such as alkaline phosphatase is applied, and the plate is incubated. After washing, an enzyme substrate, such asp-nitrophenyl phosphate, is added to the plate, and the absorbance is measured to evaluate the antigen binding activity of the sample. A fragment of the protein, such as a C-terminal fragment, may be used as a protein. BlAcore (Pharmacia) may be used to evaluate the activity of the antibody according to the present invention.

The above methods allow for the detection or measurement of the protein of the invention, by exposing the antibody of the invention to a sample assumed to contain the protein of the invention, and detecting or measuring the immune complex formed by the antibody and the protein. Because the method of detection or measurement of the protein according to the invention can specifically detect or measure a protein, the method may be useful in a variety of experiments in which the protein is used.

The present invention also provides a polynucleotide containing at least 15 nucleotides complementary to the DNA (SEQ ID NO:1, 3, 5, 7, 9, 11, 13, 15, 17, 19, 21, or 23) encoding the human KP protein or the complementary strand thereof.

Herein, the term “complementary strand” is defined as one strand of a double strand DNA composed of A:T and G:C base pair to the other strand. Also, “complementary” is defined as not only those completely matching within a continuous region of at least 15 nucleotides, but also having a homology of at least 70%, favorably 80% or higher, more favorably 90% or higher, and most favorably 95% or higher within that region. The homology may be determined using the algorithm described herein.

Such a nucleic acid includes probes and primers used for the detection and amplification of DNA encoding the inventive protein; probes and primers used for the detection of expression of the DNA; and nucleotide and nucleotide derivatives (e.g., antisense oligonucleotide and ribozyme, or DNAs encoding them, etc.) used for the regulation of expression of the inventive protein. In addition, such a nucleic acid can also be used for the preparation of DNA chip.

When used as primers, such nucleic acids are complementary at the 3′end, and restriction enzyme recognition sequences or tags can be added to the 5′end.

The antisense oligonucleotides include, for example, antisense oligonucleotides hybridizing to any region of the nucleotide sequence of SEQ ID NO:1, 3, 5, 7, 9, 11, 13, 15, 17, 19, 21, or 23. The antisense oligonucleotide is preferably an antisense of a continuous sequence of a length of 15 nucleotides or longer within the nucleotide sequence of SEQ ID NO: 1, 3, 5, 7, 9, 11, 13, 15, 17, 19, 21, or 23. More preferably, the above continuous sequence of a length of 15 nucleotides or longer contains the translation initiation codon.

A derivative or modified form of antisense oligonucleotide may also be used. The modified antisense oligonucleotides may be those modified with lower alkylphosphonate such as methylphosphonate and ethylphosphonate; phosphorothioate; phosphoroamidate; and so on.

Herein, an antisense oligonucleotide is not restricted to those in which all nucleotides are complementary to the corresponding nucleotides within a given region of a DNA or mRNA; so long as it can specifically hybridize with the nucleotide sequences of SEQ ID NO: 1, 3, 5, 7, 9, 11, 13, 15, 17, 19, 21, or 23, it may have one or more nucleotide mismatches.

A derivative of the antisense oligonucleotide of the present invention may act on cells producing the protein of the invention and may bind to a DNA or mRNA encoding the protein, whereby inhibiting the expression of the protein of the invention by inhibiting its transcription or translation, or by promoting the degradation of mRNA, and thereby inhibiting the function of the protein of the invention.

A derivative of the antisense oligonucleotide of the present invention may be mixed with appropriate carriers which are inactive against the derivative, and may be used as a medicine for externally application such as salve or poultice.

If necessary, it may be mixed with an excipient, isotonizing agent, solubilizing agent, stabilizer, preservative, pain-killer, or the like, and prepared as a tablet, powder, granule, capsule, liposome capsule, injectable solution, liquid formulation, nose drops, freeze-dried agent, etc. The above may be achieved according to standard methods.

For treating patients, a derivative of an antisense oligonucleotide of the present invention may be, for example, directly applied to the affected area of a patient, or administered into blood vessels so as to finally reach the affected area. Moreover, the derivative may be encapsulated in antisense-encapsulating materials such as liposome, poly-L-lysine, lipid, cholesterol, lipofectin, or their derivative in order to increase durability and/or membrane permeability.

Dose of the derivative of the antisense oligonucleotide of the present invention may be appropriately adjusted depending on the patient's conditions, and a favorable amount such as 0.1 to 100 mg/kg, or more preferably 0.1 to 50 mg/kg may be administered.

As the antisense oligonucleotides of the present invention inhibit expression of the protein of the invention, they find utility as inhibitors of the biological activity of the protein of the invention. An inhibitor of expression comprising the antisense oligonucleotide of the present invention is useful because it can inhibit the biological activity of the protein of the invention.

The protein of the invention may be used to screen for compounds that bind to the protein of the present invention. Specifically, the protein may be used in methods of screening for compounds, which method comprises the steps of exposing the protein of the present invention to a test sample in which a compound binding to the protein is expected to be contained; and selecting the compound having the activity of binding to the protein.

The proteins of the invention used for screening may be recombinant or natural proteins, or partial peptides. Alternatively, they may be expressed on the surface of cells or in the form of a membrane fraction. There is no particular restriction on the test sample as it includes, for example, cell extract, cell culture supernatant, product of fermentation microorganism, extract from marine organism, extract from plant, purified or crude protein, peptide, non-peptide compound, synthetic low-molecular-weight compound, natural compound, etc. The inventive protein to be contacted with a test sample can be contacted with the test sample, for example, as a purified protein, as a soluble protein, in a form of protein immobilized on carriers, as a fusion protein with other proteins, in a form of protein presented on cell membrane, as a membrane fraction.

Many methods known to those skilled in the art can be used to screen proteins capable of binding to the inventive protein. Such screening can be carried out, for example, by the immunoprecipitation method. Specifically, the method can be carried out as follows. The gene encoding a protein of this invention is expressed by inserting the gene into a vector for foreign gene expression in pSV2neo, pcDNA I, pCD8, and such, and expressing the gene in animal cells, etc. Any generally used promoters may be employed for the expression, including the SV40 early promoter (Rigby In Williamson (ed.), Genetic Engineering, Vol. 3. Academic Press, London, p.83-141 (1982)), EF-1 α promoter (Kim et al., Gene 91:217-223, 1990), CAG promoter (Niwa et al., Gene 108:193-200, 1991), RSV LTR promoter (Cullen, Methods in Enzymology 152:684-704, 1987), SR α promoter (Takebe et al., Mol. Cell. Biol. 8:466, 1988), CMV immediate early promoter (Seed et al., Proc. Natl. Acad. Sci. USA 84:3365-3369, 1987), SV40 late promoter (Gheysen et al., J. Mol. Appl. Genet. 1:385-394, 1982), Adenovirus late promoter (Kaufman et al., Mol. Cell. Biol. 9:946, 1989), HSV TK promoter, etc.

Transfer of a foreign gene into animal cells for its expression can be performed by any of the following methods, including the electroporation method (Chu et al., Nucl. Acid Res. 15:1311-1326, 1987), the calcium phosphate method (Chen et al., Mol. Cell. Biol. 7:2745-2752, 1987), the DEAE dextran method (Lopata et al., Nucl. Acids Res. 12:5707-5717, 1984; Sussman et al., Mol. Cell. Biol. 4:1642-1643, 1985), the lipofectin method (Derijard, Cell. 7:1025-1037, 1994; Lamb et al., Nature Genetics 5:22-30, 1993; Rabindran et al., Science 259:230-234, 1993), etc.

The protein of this invention can be expressed as a fusion protein having a recognition site for a monoclonal antibody by introducing the recognition site (epitope) for the monoclonal antibody, the specificity of which has been established, into the N- or C-terminus of the protein of this invention. For this purpose, commercial epitope-antibody systems can be utilized (Igaku, Experimental Medicine 13:85-90, 1995). Vectors which can express fusion proteins with the β-galactosidase, maltose-binding protein, glutathione S-transferase, green fluorescence protein (GFP), and such, via the multi-cloning site are commercially available.

There is also a report that a fusion protein may be prepared by introducing only small epitope portions consisting of several to a dozen amino acid residues so as not to change the property of the protein of the present invention by the fusion. For example, epitopes such as polyhistidine (His-tag), influenza hemagglutinin (HA), human c-myc, FLAG, Vesicular stomatitis virus glycoprotein (VSV-GP), T7 gene 10 protein (T7-tag), human herpes simplex virus glycoprotein (HSV-tag), E-tag (epitope on the monoclonal phage), and such, and monoclonal antibodies to recognize them can be utilized as the epitope-antibody system for screening proteins binding to the protein of this invention (Igaku, Experimental Medicine 13:85-90, 1995).

In immunoprecipitation, immune complexes are formed by adding these antibodies to the cell lysate prepared using suitable surfactants. The immune complex comprises a protein of this invention, a protein comprising the binding ability with the protein, and an antibody. Immunoprecipitation can be also performed by using antibodies against a protein of this invention, besides using antibodies against the above-described epitopes. An antibody to a protein of this invention can be prepared, for example, by inserting a gene encoding the protein of the invention into an appropriate expression vector of E. coli to express it in the bacterium, purifying the expressed protein, and immunizing rabbits, mice, rats, goats, chicken, and such against the purified protein. The antibody can be also prepared by immunizing the above-described animals against synthetic partial peptides of the protein of the present invention.

Immune complexes can be precipitated using, for example, Protein A Sepharose and Protein G Sepharose when the antibody is a murine IgG antibody. In addition, if a protein of this invention is prepared as a fusion protein with the epitope, such as GST, an immune complex can be formed by using a substance specifically binding to these epitopes, such as glutathione-Sepharose 4B, in the same manner as in the use of the antibody against the protein of the present invention.

Immune precipitation, in general, may be carried out according to, or following the method described in the literature (Harlow et al.: Antibodies, pp.511-552, Cold Spring Harbor Laboratory publications, New York (1988)).

SDS-PAGE is generally used for the analysis of immunoprecipitated proteins. Bound proteins can be analyzed based on the molecular weights of proteins using a gel of an appropriate concentration. In this case, although proteins bound to a protein of this invention, in general, are hardly detectable by the usual protein staining method, such as Coomassie staining and silver staining, the detection sensitivity can be improved by culturing cells in a medium containing radioisotopes, such as ³⁵S-methionine and ³⁵S-cysteine, to label proteins inside the cells, and detecting the labeled proteins. Once the molecular weight of the protein is determined, the desired protein can be purified directly from the SDS-polyacrylamide gel and can be sequenced.

In addition, proteins binding to a protein of this invention can be isolated using the West-western blotting method (Skolnik et al., Cell 65:83-90, 1991) with the protein of this invention. Namely, cDNA is isolated from cells, tissues, and organs, in which the protein binding to a protein of this invention is expected to be expressed (e.g., liver and kidney), and transferred into a phage vector (for example, λgt11, ZAP, and such) to prepare a cDNA library, which is then expressed on LB-agarose plates. The protein thus expressed is fixed on a filter; reacted with the labeled, purified protein of this invention; and plaques expressing a protein bound to a protein of this invention can be detected by the label. Methods for labeling the proteins of this invention include methods using the binding activity of biotin and avidin; methods using antibodies specifically binding to the proteins of this invention, or peptides or polypeptides fused with the protein of this invention (e.g., GST); methods using the radioisotopes; methods using fluorescence; etc.

Alternatively, in another embodiment of the method for screening of the present invention, the two-hybrid system utilizing cells may be used (Fields et al., Trends Genet. 10:286-292, 1994; Dalton et al., Cell 68:597-612, 1992; “MATCHMAKER Two-Hybrid System”, “Mammalian MATCHMAKER Two-Hybrid Assay Kit”, “MATCHMAKER One-Hybrid System” (all from Clontech), “HybriZAP Two-Hybrid Vector System” (Stratagene)). In the two-hybrid system, an inventive protein or a partial peptide thereof is fused with the SRF DNA-binding region or GAL4 DNA-binding region, and then is expressed in yeast cells; a cDNA library, which express proteins in the form of fusion protein with the VP16 or GAL4 transcription activation region, is prepared from cells that are predicted to express a protein binding to an inventive protein; the resulting cDNA library is introduced into the above-mentioned yeast cells; and then a cDNA derived from the library is isolated from a detected positive clone (when a protein binding to the inventive protein is expressed in yeast cells, the reporter gene is activated by the binding of the two proteins, and thus positive clones are detectable). A protein encoded by the cDNA can be prepared after the isolated cDNA is introduced and expressed in E coli. Thus it is possible to prepare a protein binding to an inventive protein or the encoding gene. Reporter genes to be used in the two-hybrid system include, but are not limited to, for example, Ade2 gene, LacZ gene, CAT gene, luciferase gene, PAI-1 (Plasminogen activator inhibitor typel) gene in addition to HIS3 gene. The screening by the two-hybrid method can be conduced by using mammalian cells or others in addition to yeast.

Compounds binding to a protein of the present invention can be screened by affinity chromatography. For example, a protein of the invention is immobilized on a carrier of an affinity column, and a test sample, in which a protein binding to the protein of the invention is supposed to be expressed, is applied to the column. A test sample herein may be, for example, cell extracts, cell lysates, etc. After loading the test sample, the column is washed, and proteins bound to a protein of the invention can be prepared.

The amino acid sequence of the resulting protein is then analyzed. Based on the result, an oligo-DNA is synthesized and used as the probe to screen a cDNA library. This can provide a DNA encoding the protein.

In the present invention, a biosensor on the basis of surface plasmon resonance phenomenon can be used as a means to detect or assay the bound compounds. By utilizing the biosensor on the basis of surface plasmon resonance phenomenon, the interaction between the inventive protein and a test compound can be observed as a surface plasmon resonance signal in real time using a small amount of protein without labeling (e.g., BlAcore, Pharmacia). Thus the binding between the inventive protein and the test compound can be assessed by using biosensor of BlAcore, or the like.

In addition, methods are known in the art for isolating compounds binding to a protein of the invention, which are not limited only to proteins (including agonists and antagonists). Such methods include, for example, the method of screening for a molecule binding to a protein of the invention by contacting a synthetic compound or natural substance bank, or a random phage peptide display library with an immobilized protein of the invention, and the high-throughput screening method using a combinatorial chemistry technique (Wrighton et al., Science 273:458-64, 1996; Verdine, Nature 384:11-13, 1996; Hogan, J. C. Jr., Nature 384:17-9, 1996).

Compounds isolated by the screening of this invention are candidates for agents to regulate the activity of a protein of this invention, and thought to be applied to treatments for disorders caused by expressional and functional abnormalities, and such of the protein, and diseases which can be treated by controlling the activity of the protein. Compounds which can be obtained by the screening method of this invention, the partial structure of which is modified by addition, deletion and/or substitution, are also included in the compounds binding to the protein of this invention.

When a protein of this invention or compounds isolated by the screening of this invention are used as drugs for humans and other animals, for example, mice, rats, guinea pigs, rabbits, chickens, cats, dogs, sheep, pigs, cattle, monkeys, baboons, and chimpanzees, they can be administered by directly administering the protein or isolated compound itself to a patient or by administering it after formulated according to known pharmaceutical methods. They can be administered, as the occasion demands, for example, orally, as sugar-coated tablets, capsules, elixirs and microcapsules, or parenterally, in the form of sterile solutions in water or other pharmaceutically acceptable liquids, or suspensions for injections. For example, they may be formulated by appropriately mixing with pharmaceutically acceptable carriers or media, specifically sterile water, physiological saline, plant oil, emulsifying agents, suspending agents, surfactants, stabilizers, seasonings, excipients, vehicles, anticeptics, binders, and such, in the unit dosage form required in a generally accepted pharmaceutical procedure. Amounts of effective ingredients in these pharmaceutical preparations are adjusted so as to obtain the appropriate dose in the specified range.

Additives which can be mixed in tablets and capsules include, for example, binders such as gelatin, corn starch, tragacanth gum and arabic gum; excipients such as crystalline cellulose; bulking agents such as corn starch, gelatin and alginic acid; lubricants such as magnesium stearate; sweetening agents such as sucrose, lactose or saccharine; and flavors such as peppermint, Gaultheria adenothrix oil or cherry. When the dispensing unit form is a capsule, liquid carriers, such as oil, can be further added to the above-described materials. Sterile compositions for injection can be prescribed using vehicles such as distilled water for injection according to standard pharmaceutical procedure.

Aqueous solutions for injections include, for example, physiological saline, and isotonic solutions containing: glucose and other supplements such as D-sorbitol, D-mannose, D-mannitol, sodium chloride, and such; and suitable solubilizers, for example, alcohols, more specifically, ethanol, polyalcohols such as propylene glycol, polyethylene glycol, non-ionic surfactants such as polysorbate 80 (TM) and HCO-50 may be used together.

Oily solutions, including sesame oil and soybean oil, and benzyl benzoate and benzyl alcohol may be used together as the solubilizer. Injections may be combined with buffers such as phosphate buffer and sodium acetate buffer; soothing agents such as procaine hydrochloride; stabilizers such as benzyl alcohol, phenols and antioxidants. Injections thus prepared are typically filled in suitable ampules.

The administration to patients is done by methods commonly known to those skilled in the art, such as intraarterial, intravenous, or subcutaneous injections, as well as intranasal, bronchial, intramuscular, percutaneous, or oral administrations. One skilled in the art can suitably select the dosage according to the body-weight or age of a patient, or the method of administration. If the compound can be encoded by DNA, the DNA may be used for gene therapy by incorporating the DNA into a vector for gene therapy. Dosages and administration methods vary depending on the body-weight, age, symptoms, and such of patients, but those skilled in the art can appropriately select them.

Although the specific dosage of the protein of the invention changes according to the subject to be treated, the target organs, symptoms, and administration methods, it is generally considered to be, for example, about 100 μg to 20 mg one day for an adult (as body-weight 60 kg) in the form of injections.

Though they vary depending on the symptoms, doses of compounds binding to a protein of this invention or compounds regulating the activity of such a protein may be generally in the range of about 0.1 to 100 mg, preferably about 1.0 to 50 mg, and more preferably about 1.0 to 20 mg per day for adults (based on the body weight 60 kg) in the case of oral administration.

Though it varies depending on the subject to be administered, target organ, symptom and method of administration, a single dose of the compounds for the parenteral administration is thought to be preferably administered, for example, when it is in the form of injection, intravenously to normal adults (based on the body weight 60 kg) in the range of about 0.01 to 30 mg, preferably about 0.1 to 20 mg, and more preferably about 0.1 to 10 mg or thereabout per day. Doses converted on the 60 kg body weight basis or the body surface area can be similarly administered to other animals.

All publications and patents cited herein are incorporated by reference in their entirety.

### DETAILED DESCRIPTION

The invention is illustrated more specifically with reference to the following examples, but is not to be construed as being limited thereto.

### EXAMPLE 1

Construction of a CDNA library by the oligo-capping method

The NT-2 neuron progenitor cells (Stratagene), teratocarcinoma cells from human fetal testis, which can be differentiated into neurons by the treatment with retinoic acid were treated as follows according to the manufacturer's instructions.

(1) NT-2 cells were cultured without the induction by retinoic acid (NT2RM4).

(2) After NT-2 cells were cultured, retinoic acid was added thereto, and the cells were cultured for 2 weeks (NT2RP2).

After the culture, the respective cells were collected, and mRNA was extracted according to the method described in the literature (Sambrook et al., Molecular Cloning Second edition, Cold Spring harbor Laboratory Press 1989). Then, poly(A)⁺RNA was purified by using oligo dT cellulose.

Similarly, human placenta (PLACE1, PLACE2), human ovary cancer tissue (OVARC1), and tissue from human embryo at 10 weeks, which is enriched with head (HEMBA1) were used to extract mRNA by the method described in the literature (Molecular Cloning 2nd edition. (1989) Sambrook et al., Cold Spring Harbor Laboratory Press). Furthermore, poly(A)⁺RNA was purified from the mRNA using oligo-dT cellulose.

This poly(A)⁺RNA was used to construct a cDNA library by the oligo-capping method (Maruyama et al., Gene 138:171-174, 1994). Using the Oligo-cap linker (agcaucgagu cggccuuguu ggccuacugg/ SEQ ID NO:26) and the Oligo-dT primer (gcggctgaag acggcctatg tggccttttt tttttttt tt/ SEQ ID NO:27), bacterial alkaline phosphatase (BAP) treatment, tobacco acid phosphatase (TAP) treatment, RNA ligation, the first strand cDNA synthesis, and removal of RNA were performed according to the references (Suzuki et al., Protein, Nucleic acid and Enzyme, 41:197-201, 1996; Suzuki et al., Gene 200:149-156, 1997). Then, 5′- and 3′-PCR primers (agcatcgagt cggccttgtt g/ SEQ ID NO:28, and gcggctgaag acggcctatg t/ SEQ ID NO:29, respectively) were used for performing PCR to convert the cDNA into double stranded cDNA, which was then digested with SfiI. Then, the DraIII-cleaved vector pUC19FL3 or pME18SFL3 (GenBank AB009864, expression vector) (NT2RM4, NT2RP2, NT2RP3, PLACE1, PLACE2, OVARC 1, HEMBA 1) was used for cloning the CDNA in a unidirectional manner, and cDNA libraries were obtained. The nucleotide sequence of the 5′- and 3′- ends of the cDNA clones was analyzed with a DNA sequencer (ABI PRISM 377, PE Biosystems) after sequencing reactions performed with the DNA sequencing reagents (Dye Terminator Cycle Sequencing FS Ready Reaction Kit, dRhodamine Terminator Cycle Sequencing FS Ready Reaction Kit, or BigDye Terminator Cycle Sequencing FS Ready Reaction Kit, PE Biosystems), according to the instructions. The obtained data were used for a database.

Oligo-cap high full-length ratio cDNA libraries for samples except NT2RM1 and NT2RP1 were prepared by using an expression vector, pME18SFL3, which can be expressed in eukaryotic cells. pME18SFL3 vector contains the SRα. promoter and SV40 small t intron in the upstream, as well as the SV40 polyA addition signal sequence downstream of the cloning site, respectively. As the cloning site of pME18SFL3 has asymmetrical DraIII sites, and the ends of cDNA fragments contain SfiI sites complementary to the DraIII sites, the cloned cDNA fragments can be unidirectionally inserted downstream of the SRα promoter. Therefore, clones containing full-length cDNA can be expressed transiently by introducing the obtained plasmid directly into COS cells. Thus, the clones can be analyzed very easily in terms of the proteins that are the gene products of the clones, or in terms of the biological activities of the proteins.

### EXAMPLE 2

Estimation of the completeness at the 5′-ends of the clones contained in the cDNA libraries constructed by the oligo-capping method

The full-length ratio at the 5′-end sequence of respective clones in the human cDNA libraries constructed by the oligo-capping method was determined as follows. The clones whose 5′-end sequences were consistent with those of known human mRNA in the public database were judged to be “full-length” if they had a longer 5′-end sequence than that of the known human mRNA; or even though the 5′-end sequence was shorter, if it contained the translation initiation codon it was judged to have the “full-length” sequence. Clones which did not contain the translation initiation codon were judged to be “not-full-length”. The full-length ratio ((the number of full-length clones)/(the number of full-length and not-full-length clones)) at the 5′-end of the cDNA clones from each library was determined by comparing with known human mRNA. As a result, the full-length ratio of the 5′-ends was 63.5%. The result indicates that the full-length ratio at the 5′-end sequence was extremely high in the human cDNA clones obtained by the oligo-capping method.

### EXAMPLE 3

Assessment of the full-length ratio of the 5′-end of the cDNA by the ATGpr and the ESTiMateFL

The ATGpr, developed by Salamov A. A., Nishikawa T., and Swindells M. B. in the Helix Research Institute, is a program for prediction of the translation initiation codon based on the characteristics of the sequences in the vicinity of the ATG codon (Salamov et al., Bioinformatics 14:384-390, 1998; http://www.hri.cojp/atgpr/). The results are shown with expectations (also mentioned as ATGprl below) whether the ATG is a true initiation codon (0.05-0.94). When it was not considered that the sequence was the 5′-end of the cDNA or not, both of the sensitivity and specificity of analytical results by this program were estimated as 66%. When the program was applied to the 5′-end sequences of the clones from the cDNA library that was obtained by the oligo-capping method having 65% full-length ratio, the sensitivity and specificity of the estimation of the full-length clone (clone containing the N-terminus of the ORF) were improved to 82 to 83% by selecting only clones having an ATGprl score 0.6 or higher. The maximum ATGpr1 scores for 5′-end sequences were shown in Table 1.

TABLE 1

| Clone name | Sequence name | Maximum ATGpr1 score |
|---|---|---|
| HEMBA1002212 | F-HEMBA1002212 | 0.39 |
| HEMBA1006173 | F-HEMBA1006173 | 0.42 |
| NT2RM4001411 | F-NT2RM4001411 | 0.47 |
| NT2RM4001758 | F-NT2RM4001758 | 0.59 |
| NT2RP2000668 | F-NT2RP2000668 | 0.81 |
| NT2RP2001839 | F-NT2RP2001839 | 0.83 |
| NT2RP2002710 | F-NT2RP2002710 | 0.94 |
| NT2RP2004933 | F-NT2RP2004933 | 0.94 |
| PLACE1011923 | F-PLACE1011923 | 0.74 |
| PLACE2000034 | F-PLACE2000034 | 0.88 |
| OVARC1000556 | F-OVARC1000556 | 0.94 |
| HEMBA1001019 | F-HEMBA1001019 | 0.08 |

Next, the ESTiMateFL was used for the assessment of the clones. The ESTiMateFL, developed by Nishikawa and Ota in the Helix Research Institute, is a method for selecting clones expected to have a full-length cDNA by comparing with the 5′-end or 3′-end sequences of ESTs in the public database.

By this method, a cDNA clone is judged to be most likely not to be full-length if there exist any ESTs which have longer 5′-end or 3′-end sequences than the clone. The method is systematized for high throughput analysis. A clone is judged to be full-length if the clone has a longer 5′-end sequence than the ESTs in the public database corresponding thereto. Even if a clone has a shorter 5′-end, the clone is judged to be full-length if the difference in length is within 50 bases, and otherwise judged not to be full-length, for convenience. Those clones whose 5′-end sequence is matching with the known MRNA, about 80% of the clones judged to be full-length by the comparison with ESTs were also judged to be full-length by the assessment of the 5′-end sequence by comparing with known mRNA. Also, about 80% of the clones judged to be not full-length in the 5′-end sequence by comparing with ESTs were also judged to be not full-length in the 5′-end sequence by comparison with known mRNA. The precision of the estimation by comparing with ESTs is improved with increasing numbers of ESTs to be compared. However, in case with limited numbers of ESTs, the reliability becomes low. Thus, the method is effective in excluding clones with high probability of being not-full-length from the cDNA clones that is synthesized by the oligo-capping method having a 5′-end sequence full-length ratio of about 60%. In particular, the ESTiMateFL is efficiently used in estimating the full-length ratio at the 3′-end sequence of cDNA of a human unknown mRNA, a significant number of which are deposited in the public database as EST deposits.

Results of the above assessment for the full-length ratio showed that the clones “C-HEMBA1006173”, “C-PLACE2000034”, and “OVARC1000556” were novel clones with a high probability of being full-length and also which share no sequence identity with any of human EST sequences at least either at the 5′-end sequence or 3′-end sequence, or both ends.

Whereas the “C-HEMBA1002212” is a full-length sequence and is a novel clone; the number of human EST sequences that shared a common sequence to this clone at both 5′-and 3′-end was 1 or more and 5 or less.

Furthermore, “C-NT2RM4001411 ”, “C-NT2RM4001758”, “C-NT2RP2000668”, “C-NT2RP2001839”, “C-NT2RP2002710”, “C-NT2RP2004933”, and “C-PLACE1011923” are also full-length clones; the number of human EST sequences that shared a common sequence to each of these clones at the 5′-end was 20 or less (clones which do not share sequences with certain human EST sequences at least either at the 5′-end or at 3′-end, or at both ends of the clone; excluding clones in which the number of human EST sequences that shared a common sequence to each of the clones at both of the 5′- and 3-end was 1 or more and 5 or less). Accordingly, they were concluded to be novel clones.

Although the clone “C-HEMBAl001019” has a low ATGpr1 value and a low ATGpr2 value, still it was a full-length clone based on the full-length ratio, and furthermore, it is a novel clone which shares no sequence identity at least at the 5′-end with any human EST sequences.

### EXAMPLE 4

Selection of clones having a kinase/phosphatase-like sequence

Clones having a kinase/phosphatase-like sequence were selected from the helix clones. All the helix clones were searched for homology by NCBI TBLASTN2.0 by using the following 31 amino acid sequences of known kinases and phosphatases (also including phospholipid kinases) as queries. Clones with a expectation value (Expect) 1.0e-05 or lower were selected.

The query sequences used in the homology search as well as their SEQ ID NOs and GenBank accession numbers are as follows.

| Query sequence No. | SEQ ID NO: | GenBank accession No. |
|---|---|---|
| hLKB1 | 30 | gi\|3024670 |
| hVRK1 | 31 | gi\|4507903 |
| hCDC2 | 32 | gi\|4502709 |
| hAuroraK1 | 33 | gb\|AAC1278.1 |
| hAuroraK2 | 34 | gi\|4759178 |
| hIKKA | 35 | gb\|AAC51662.1 |
| hMKK3 | 36 | gb\|AAB40653.1 |
| hERK1 | 37 | pir\|A48082 |
| hRAF1 | 38 | gi\|4506401 |
| hAKT | 39 | gi\|4885061 |
| hPIKP85 | 40 | sp\|P27986 |
| hATM | 41 | gi\|4502267 |
| hc-src | 42 | gi\|4758078 |
| hJAK1 | 43 | ref\|NP_002218.1 |
| hFLT1 | 44 | gb\|AAC16449.1 |
| hPP2A | 45 | gi\|4506017 |
| hMKP2 | 46 | gb\|AAC50452.1 |
| hVHR | 47 | gi\|4758208 |
| hPTP-SL | 48 | gi\|4506325 |
| hSTEP | 49 | sp\|P54829 |
| hPTEN | 50 | gi\|4506249 |
| Cdc14B1 | 51 | gb\|AAD15415.1 |
| DUSP12 | 52 | gi\|6005956 |
| AK000449 | 53 | gi\|8923413 |
| DUS7 | 54 | sp\|Q16829 |
| calcineurin A alpha | 55 | gi\|6715568 |
| PNP1 | 56 | emb\|CAA56124.1 |
| TPTE | 57 | gi\|7019559 |
| PPP1CC | 58 | gi\|4506007 |
| PP-1 gamma | 59 | gb\|AAA19823.1 |
| PP2A | 60 | gi\|4506017 |

The results of homology search were shown in Tables 2 and 3.

TABLE 2

| | | Search score | Expectation |
|---|---|---|---|
| Query | Helix clone | (score) | value (expect) |
| hLKB1 | C-NT2RP2004933 | 126 | 1e-29 |
| hLKB1 | C-PLACE1011923 | 89 | 2e-28 |
| hLKB1 | C-NT2RM4001758 | 118 | 3e-27 |
| hLKB1 | C-OVARC1000556 | 64 | 1e-10 |
| hCDC2 | C-NT2RP2004933 | 109 | 1e-24 |
| hCDC2 | C-HEMBA1001019 | 72 | 3e-13 |
| hCDC2 | C-NT2RM4001758 | 68 | 5e-12 |
| hCDC2 | C-OVARC1000556 | 53 | 1e-07 |
| hCDC2 | C-PLACE1011923 | 47 | 6e-06 |
| hAuroraK1 | C-PLACE1011923 | 115 | 3e-37 |
| hAuroraK1 | C-NT2RP2004933 | 145 | 2e-35 |
| hAuroraK1 | C-NT2RM4001758 | 121 | 3e-28 |
| hAuroraK1 | C-NT2RP2000668 | 66 | 2e-11 |
| hAuroraK1 | C-NT2RP2001839 | 53 | 2e-07 |
| hAuroraK1 | C-OVARC1000556 | 51 | 6e-07 |
| hAuroraK2 | C-PLACE1011923 | 105 | 4e-37 |
| hAuroraK2 | C-NT2RP2004933 | 138 | 2e-33 |
| hAuroraK2 | C-NT2RM4001758 | 112 | 1e-25 |
| hAuroraK2 | C-OVARC1000556 | 57 | 9e-09 |
| hIKKA | C-NT2RP2004933 | 103 | 2e-22 |
| hIKKA | C-NT2RM4001758 | 82 | 5e-16 |
| hIKKA | C-PLACE1011923 | 48 | 8e-06 |
| hMKK3 | C-PLACE1011923 | 75 | 4e-14 |
| hNKK3 | C-NT2RP2004933 | 63 | 1e-10 |
| hNKK3 | C-NT2R144001758 60 | 9e-10 | |
| hMKK3 | C-HEMBA1002212 | 60 | 1e-09 |
| hERK1 | C-NT2RP2004933 | 89 | 2e-18 |
| hERK1 | C-PLACE1011923 | 70 | 1e-12 |
| hERK1 | C-NT2RM4001758 | 60 | 1e-09 |
| hERK1 | C-OVARC1000556 48 | 4e-06 | |

(TABLE 3)

| | | Search score | Expectation |
|---|---|---|---|
| Query | Helix clone | (score) | value (expect) |
| hRAF1 | C-NT2RP2004933 | 73 | 2e-13 |
| hRAF1 | C-PLACE1011923 | 72 | 6e-13 |
| hRAF1 | C-NT2RM4001758 | 65 | 8e-11 |
| hAXT | C-NT2RM4001758 | 129 | 1e-30 |
| hAKT | C-NT2RP2004933 | 129 | 1e-30 |
| hART | C-PLACE1011923 | 97 | 9e-28 |
| hART | C-OVARC1000556 | 60 | 2e-09 |
| hPIKP85 | C-N72RP2002710 | 65 | 7e-11 |
| hc-src | C-NT2RM4001758 | 69 | 2e-12 |
| hc-src | C-NT2RP2004933 | 65 | 5e-11 |
| hc-src | C-NT2RM4001411 | 58 | 7e-09 |
| hc-src | C-HEMBA1002212 | 57 | 1e-08 |
| hJAK1 | C-NT2RP2004933 | 82 | 7e-16 |
| hJAK1 | C-REMBA1002212 | 60 | 5e-09 |
| hJAK1 | C-NT2RM4001758 | 55 | 2e-07 |
| hFLT1 | C-HEMBA1002212 | 64 | 2e-10 |
| hFLT1 | C-NT2RP2004933 | 59 | 7e-09 |
| hFLT1 | C-PLACE1011923 | 55 | 1e-07 |
| hFLT1 | C-PLACE2000034 | 54 | 2e-07 |
| hFLT1 | C-NT2RM4001758 | 51 | 2e-06 |
| hSTEP | C-HEMBA1006173 | 51 | 1e-06 |

Based on the result, non-overlapping 12 clones were selected as clones having kinase/phosphatase-like structure (KP clones), which are “C-NT2RP2000668”, “C-HEMBA002212”, “C-NT2RM4001411”, “C-NT2RM4001758”, “C-NT2RP2002710”, “C-NT2RP2004933”, “C-PLACE 1011923”, “C-NT2RP2001839”, “C-HEMBA1006173”, “C-OVARC1000556”, “C-PLACE2000034”, and “C-HEMBA1001019”. The clones encode novel human proteins, and each of the proteins was deduced to function as a protein kinase and/or a protein phosphatase.

### EXAMPLE 5

Gene expression analysis by hybridization using high density DNA filter

DNA for spotting onto the nylon membranes was prepared according to the following procedure. E. coli was cultured in each well of a 96-well plate (in a LB medium at 37° C. for 16 hours). A part of each culture was suspended in 10 μl of sterile water in the well of a 96-well plate. The plate was heated at 100° C. for 10 minutes. Then the samples were analyzed by PCR. PCR was performed in a 20 μl solution per one reaction by using TaKaRa PCR Amplification Kit (Takara) according to the supplier's protocol. A pair of sequencing primers, ME761 FW (5′tacggaagtgttacttctgc 3′/ SEQ ID NO:61) and ME1250RV (5′tgtgggaggttttttctcta 3′/SEQ ID NO:62), or a pair of primers, M13M4 (5′gttttcccagtcacgac 3′/SEQ ID NO:63) and M13RV (5′caggaaacagctatgac 3′/SEQ ID NO:64) were used for the amplification of the insert cDNA in the plasmid. PCR was performed in a thermal cycler, GeneAmp System 9600 (PE Biosystems). The cycling profile consisted of pre-heating at 95° C. for 5 minutes; 10 cycles of denaturation at 95° C. for 10 seconds, and annealing/extension at 68° C. for 1 minute; 20 cycles of denaturation at 98° C. for 20 seconds and annealing/extension at 60° C. for 3 minutes; and final extension at 72° C. for 10 minutes. After the PCR, 2 μl of the reaction solution was electrophoresed on a 1% agarose gel. DNA on the gel was stained with ethidium bromide to confirm the amplification of cDNA. When cDNAs were not amplified by PCR, plasmids containing the corresponding insert cDNAs were prepared by the alkali-extraction method (Sambrook et al., Molecular Cloning, A laboratory manual/ 2nd edition, Cold Spring Harbor Laboratory Press, 1989).

DNA array was prepared by the following procedure. An Aliquot of the DNA solution was added to each well of a 384-well plate. DNA was spotted onto a nylon membrane (Boehringer) by using a 384-pin tool of Biomek 2000 Laboratory Automation System (Beckman-Coulter). More specifically, the 384-well plate containing the DNA was placed under the 384-pin tool. The independent 384 needles of the pin tool were simultaneously dipped into the DNA solution to fix the DNA on the needles. The needles were gently pressed onto a nylon membrane, and the DNA fixed on the needles was spotted onto the membrane. Denaturation of the spotted DNA and immobilization of the DNA on the nylon membrane were carried out according to conventional methods (Sambrook et al., Molecular Cloning, A laboratory manual/2nd edition, Cold Spring Harbor Laboratory Press, 1989).

1st strand cDNA labeled with radioisotope was used as the hybridization probe. The 1st strand cDNA was synthesized by using Thermoscript(^{M}) RT-PCR System (GIBCO). More specifically, the 1st strand cDNA was synthesized by using 1.5 μg mRNAs from various human tissues (Clontech), 1 μl 50 μM Oligo(dT)20, and 50 μCi [α³³P]dATP according to the attached protocol. Purification of the probe was carried out by using ProbeQuant(™) G-50 micro column (Amersham-Pharmacia Biotech) according to the attached protocol. In the next step, 2 units of E. coli RNaseH were added to the reaction mixture. The mixture was incubated at room temperature for 10 minutes, and then 100 μg of human COT-1 DNA (GIBCO) was added thereto. The mixture was incubated at 97° C. for 10 minutes, and then was allowed to stand on ice to give the hybridization probe.

Hybridization of the radioisotope-labeled probe to the DNA array was performed in a usual manner (Sambrook et al., Molecular Cloning, A laboratory manual/ 2nd edition, Cold Spring Harbor Laboratory Press, 1989). The membrane was washed as follows: the nylon membrane was washed three times by incubating in the Washing solution 1 (2X SSC, 1% SDS) at room temperature (about 26° C.) for 20 minutes; then the membrane was washed 3 times by incubating it in the Washing solution 2 (0.1X SSC, 1% SDS) at 65° C. for 20 minutes. Autoradiography was performed by using an image plate for BAS2000 (Fuji Photo Film Co., Ltd.). Specifically, the nylon membrane used for the hybridization was wrapped with a piece of Saran Wrap, and was contacted with the light-sensitive surface of the image plate. The membrane with the image plate was placed in an imaging cassette for radioisotope and was allowed to stand in dark for 4 hours. The radioactivity recorded on the image plate was analyzed by BAS2000 (Fuji Photo Film Co., Ltd.) and was recorded as an image file of the autoradiogram by electronic conversion. The signal intensity of each DNA spot was analyzed by using Visage High Density Grid Analysis Systems (Genomic Solutions Inc.). The signal intensity was converted into numerical data. The data were taken by duplicated measurements. The reproducibility was assessed by comparing the signal intensities of the corresponding spots on the duplicated DNA filters that were hybridized to a single DNA probe. The ratio between the corresponding spots falls within a range of 2-folds or less in 95% of entire spots, and the correlation coefficient was r=0.97. Thus, the reproducibility was assumed to be satisfactory.

The detection sensitivity in gene expression analysis was estimated by examining increases in the signal intensity of the probe concentration-dependent spot of the hybridization using a probe complementary to the DNA spotted on the nylon membrane. PLACE1008092 (the same DNA as that deposited in GenBank Accession No. AF107253) was used as the DNA. The DNA array with the DNA of PLACE1008092 was prepared according to the above-mentioned method. The probe was prepared as follows: mRNA was synthesized in vitro from the clone, PLACE1008092; using this mRNA as the template, radioisotope-labeled 1st strand cDNA was synthesized in the same manner as the probe preparation method described above; and the cDNA was used as the probe. The cDNA PLACE1008092 was inserted into pBluescript SK(-), so that the 5′-end of the PLACE1008092 is ligated to the T7 promoter of the pBluescript SK(-) to give a recombinant plasmid for in vitro synthesis of the mRNA from PLACE1008092. Specifically, the PLACE1008092 inserted at the DraIII site of the pME18SFL3 was cut out by XhoI digestion. The resulting PLACE1008092 fragment was ligated to XhoI-predigested pBluescript SK(-) by using the DNA ligation kit ver.2 (Takara). The in-vitro mRNA synthesis from PLACE1008092 inserted in pBluescript SK(-) was carried out by using the Ampliscribe(™) T7 high yield transcription kit (Epicentre technologies). The hybridization and analysis of signal intensity of each DNA spot were conducted using the same methods described above. When the probe concentration was 1×10⁷ μg/ml or less, there was no increase of signal intensity proportional to the probe concentration. Therefore it was assumed to be difficult to compare the signals with one another in this concentration range. Thus, spots with a intensity of 40 or less were indiscriminately taken as low-level signals. Within a concentration of the probe ranging from 1×10⁷ μg/ml to 0.1 μg/ml, signals were found to increase in a probe concentration-dependent manner. The detection sensitivity is 1:100,000 in a ratio of mRNA expression level in a sample.

Table 4 shows the expression of each cDNA in human normal tissues (heart, lung, pituitary gland, thymus, brain, kidney, liver and spleen). The expression levels are indicated by numerical values of 0 to 10,000. Each of the “C-HEMBA1006173”, “C-NT2RP2000668”, “C-NT2RP2001839”, “C-NT2RP2002710”, “C-NT2RP2004933”, “C-OVARC 1000556”, “C-PLACE1101923”, and “C-PLACE2000034” was expressed in at least one tissue.

The expression of “C-NT2RP2002710” was detectable in all the tissues. The clones “C-HEMBA1001019”, “C-HEMBA1002212”, “C-NT2RM4001411”, and “C-NT2RM4001758” were expressed at only low levels in all the tissues.

Further, genes expressed in a characteristic way were selected by statistical analysis of the data. Genes which expression level greatly varies among tissues as compared with the expression level of β-actin commonly used as a control, were identified as follows. Specifically, the sum of squared deviation of signal intensity in each tissue was determined for β-actin, and then, the value was divided by the degree of freedom 7 to determine variance Sₐ^{2.} Next, the sum of squared deviation of signal intensity in each tissue was determined for the gene to be compared, and the value was divided by the degree of freedom 7 to determine variance S_{b}² Genes with significance levels 5% or higher in the F-distribution were selected taking the variance ratio as F=S_{b}^{2/}Sₐ^{2.} Thus, “C-NT2RP2002710” was selected as a gene exhibiting a characteristic feature in the expression.

Additionally, “C-NT2RP2002710” was selected as a gene which expression level greatly varied among tissues. Specifically, the sum of squared deviation of signal intensity in each tissue was determined for OVARC1000037, and the value was divided by the degree of freedom 7 to determine variance Sₐ^{2.} Next, the sum of squared deviation of signal intensity in each tissue was determined for the gene to be compared, and the value was divided by the degree of freedom 7 to determine variance S_{b}². Genes with a significance levels 5% or higher in the F-distribution, were selected taking the variance ratio as F=S_{b}²/Sₐ² .

TABLE 4

| | | | Pituitary | | | | | |
|---|---|---|---|---|---|---|---|---|
| Clone name | Heart | Lung | gland | Thymus | Brain | Kidney | Liver | Spleen |
| GAPDH | 38.210 | 32.670 | 23.820 | 13.580 | 11.230 | 21.120 | 24.910 | 22.440 |
| β-actin | 279.280 | 368.870 | 111.100 | 117.500 | 92.880 | 114.650 | 82.990 | 256.790 |
| HEMBA1001019 | 7.327 | 7.582 | 14.865 | 6.154 | 10.598 | 5.643 | 3.920 | 7.188 |
| HEMBA1002212 | 8.709 | 6.241 | 10.946 | 9.855 | 2.602 | 5.864 | 5.366 | 4.214 |
| HEMBA1006173 | 63.939 | 35.393 | 52.598 | 22.894 | 32.403 | 35.413 | 40.872 | 67.870 |
| NT2RM4001411 | 8.505 | 7.030 | 30.358 | 2.388 | 3.324 | 0.962 | 1.969 | 1.931 |
| NT2RM4001758 | 24.391 | 23.518 | 27.924 | 5.579 | 12.781 | 14.153 | 7.027 | 6.943 |
| NT2RP2000668 | 41.209 | 35.434 | 46.568 | 16.251 | 14.705 | 25.339 | 29.016 | 11.020 |
| NT2RP2001839 | 68.237 | 44.006 | 65.237 | 21.186 | 23.824 | 37.874 | 35.524 | 54.235 |
| NT2RP2002710 | 876.030 | 389.806 | 785.892 | 246.642 | 312.053 | 990.051 | 876.290 | 401.334 |
| NT2RP2004933 | 18.660 | 21.000 | 61.644 | 10.893 | 8.184 | 31.855 | 24.143 | 11.593 |
| OVARC1000556 | 84.636 | 47.645 | 29.302 | 13.010 | 36.300 | 34.811 | 56.871 | 26.716 |
| PLACE1011923 | 32.608 | 43.041 | 19.701 | 8.083 | 15.625 | 16.742 | 22.157 | 29.615 |
| PLACE2000034 | 42.847 | 21.194 | 15.709 | 12.449 | 11.089 | 18.174 | 25.238 | 21.354 |

### EXAMPLE 6

Analysis of disease-associated genes

Non-enzymic protein glycation reaction is believed to be a cause of a variety of chronic diabetic complications. Accordingly, genes of which expression is elevated or decreased in a glycated protein-specific manner are associated with diabetic complications caused by glycated proteins. Vascular endothelial cells are affected with glycated proteins present in blood. Reaction products of non-enzymic protein glycation include amadori compound (glycated protein) as a mildly glycated protein and advanced glycation endproduct as a heavily glycated protein. Hence, whether or not the expression of the KP genes of this invention was varied depending on the presence of these proteins in endothelial cells was examined.

The mRNAs were extracted from endothelial cells that were cultured in the presence or absence of glycated protein. The mRNAs were converted into radiolabeled first strand cDNAs for preparing probes. The probes were hybridized to the above-mentioned DNA array. Signal of each DNA spot was detected by BAS2000 and analyzed by ArrayGauge (Fuji Photo Film Co., Ltd.).

Advanced glycation endproduct of bovine serum albumin was prepared as follows: bovine serum albumin (BSA; Sigma) was incubated in a phosphate buffer solution containing 50 mM glucose at 37° C. for 8 weeks; and the resulting brownish BSA was dialyzed against a phosphate buffer solution.

Human normal pulmonary arterial endothelial cells (Cell Applications) were cultured in an Endothelial Cell Growth Medium (Cell Applications). The culture dish (Falcon) with the cells was incubated in a CO₂ incubator (37° C., 5% CO₂, in a humid atmosphere). When the cells were grown to be confluent in the dish, 250 μg/ml of bovine serum albumin (sigma), glycated bovine serum albumin (Sigma) or advanced glycation endproduct of serum albumin was added thereto and the cells were incubated for 33 hours. The mRNA was extracted from the cells by using a FastTrack(™) 2.0 kit (Invitrogen). The labeling of hybridization probe was carried out by using the mRNA according to the same procedure as described above.

Table 5 shows the expression level of each CDNA in human pulmonary arterial endothelial cells cultured in a medium containing bovine serum albumin, glycated bovine serum albumin or advanced glycation endproduct of bovine serum albumin. Genes of which expression was detected in the endothelial cell are “C-HEMBA1006173”, “C-NT2RP2000668”, “C-NT2RP2002710”, “C-NT2RP2001839”, “C-OVARC1000556”, “C-PLACE1011923”, and “C-PLACE2000034”.

TABLE 5

| | | | Advanced | | |
|---|---|---|---|---|---|
| | | | glycation | Glycated bovine | Advanced glycation |
| | Bovine | Glycated | endproduct of | albumin addition/ | endproduct of bovine |
| | serum | bovine | bovine serum | Bovine serum | serum albumin/Bovine |
| Clone name | albumin | albumin | albumin | albumin ratio | serum albumin ratio |
| GAPDH(Cr1) | 100.81 | 134.21 | 115.16 | 1.33 | 1.14 |
| β-actin(Cr2) | 1101.9 | 1092.57 | 997.36 | 0.99 | 0.91 |
| HEMBA1006173 | 769.5 | 879.52 | 703.94 | 1.14 | 0.91 |
| NT2RP2000668 | 100.83 | 114.72 | 104.66 | 1.14 | 1.04 |
| NT2RP2001839 | 1191.8 | 1454.46 | 831.23 | 1.22 | 0.7 |
| NT2RP2002710 | 305.22 | 324.01 | 248.69 | 1.06 | 0.81 |
| OVARC1000556 | 68.77 | 93.52 | 73.85 | 1.36 | 1.07 |
| PLACE1011923 | 318.75 | 346.7 | 273.3 | 1.09 | 0.86 |
| PLACE2000034 | 57.78 | 58.66 | 54.29 | 1.02 | 0.94 |

### EXAMPLE 7

Analysis of ultraviolet radiation damage-associated genes

It is known that ultraviolet rays give considerably adverse influence on health. In recent years, the risks of tissue damage by ultraviolet rays has been increased due to the destruction of the ozone layer, and ultraviolet radiation has been recognized as a risk factor for diseases such as skin cancers (United States Environmental Protection Agency: Ozone Depletion Home Page, http://www.epa.gov/ozone/). Genes whose expression levels change with exposure of the skin epidermal cells to ultraviolet rays are considered to be associated with skin damage caused by ultraviolet radiation. Culturing primary cultured skin fibroblast cells irradiated with ultraviolet ray, it was examined whether the expression of KP genes of this invention varies depending on the irradiation of ultraviolet ray.

First, after culturing to confluence in a culture dish, the primary cultured skin fibroblast cells (Cell Applications) were exposed to 10,000 μJ/cm² of 254-nm ultraviolet light. Thereafter, messenger RNAs were extracted by using a FastTrack™2.0 mRNA Isolation kit (Invitrogen) from the unexposed cells and from the cells that were exposed to the ultraviolet light and then cultured for 4 or 24 hours. The labeling of the hybridization probe was carried out by using 1.5 μg of each mRNA in the same manner as described above. The data were obtained in triplicate (n=3). The hybridization signals were compared between the cells exposed to the ultraviolet light and the unexposed cells. The comparison was preformed by statistical treatment with two-sample t-test. Clones with significant differences in the signal distribution were selected under the condition of p<0.05. According to the analysis, the difference in the signal values can be also detected statistically even when the signal values are low. Accordingly, clones with signal value of 40 or lower were also assessed.

Table 6 shows the expression of each cDNA in skin-derived fibroblast cells exposed and unexposed to ultraviolet light.

Averaged signal values (M₁, M₂) and sample variances (s₁², S₂²) were calculated for each gene in each of the cells, and then, pooled sample variances s² were obtained from the sample variances of the two types of cells to be compared. The t values were determined according to the following formula: t=(M₁-M₂)/s/(⅓+⅓)^{½}. When the determined t-value was greater than a t-value at P, probability of significance level, of 0.05 or 0.01 in the t-distribution table with 4 degrees of freedom, it was judged that a difference exists in the expression level of the gene between the two types of cells at P<0.05 or P<0.01, respectively. The table also includes the information of an increase (+) or decrease (−) in the average expression level of a signal in the clones compared with that of undifferentiated cells.

The results showed that the expression level of “C-NT2RP2002710”, “C-NT2RP2004933”, “C-OVARC1000556”, and “C-PLACE1011923” was reduced 4 hours or 24 hours after ultraviolet ray irradiation, suggesting that they are clones associated with ultraviolet ray disorders.

TABLE 6

| | UV_0 h | | | UV_4 h | | | UV_24 h | | | t test | | 4 h | 24 h |
|---|---|---|---|---|---|---|---|---|---|---|---|---|---|
| Clone | Exp. 1 | Exp. 2 | Exp. 3 | Exp. 1 | Exp. 2 | Exp. 3 | Exp. 1 | Exp. 2 | Exp. 3 | 0/4 | 0/24 | +/− | +/− |
| GAPDH(Cr1) | 0 | 1.29 | 0.1 | 0.9 | 0.06 | 1.18 | 1.49 | 0.47 | 0 | | | | |
| β actin(Cr2) | 256.82 | 283.53 | 414.29 | 388.38 | 117.29 | 329.8 | 189.18 | 190.26 | 157.87 | | * | | − |
| NT2RP2002710 | 30.61 | 29.84 | 37.75 | 21.5 | 19.35 | 13.67 | 11.45 | 15.23 | 13.68 | * | ** | − | − |
| NT2RP2004933 | 6.78 | 7.42 | 14.62 | 6.21 | 3.1 | 2.23 | 3.41 | 1.97 | 1.54 | | * | | − |
| OVARC1000556 | 22.37 | 23.81 | 26.15 | 14.72 | 11.54 | 9.38 | 19.99 | 16.63 | 22.58 | ** | | − | |
| PLACE1011923 | 166.86 | 139.43 | 146.9 | 104.2 | 117.42 | 86.95 | 136.93 | 140 | 124.61 | * | | − | |

### INDUSTRIAL APPLICABILITY

The present invention provides novel human protein kinase and protein phosphatase proteins, as well as genes encoding the proteins. The regulation of the phosphorylation state of proteins by kinase and/or phosphatase plays central roles in normal differentiation and/or proliferation of cells, as well as in physiological functions at the cellular level. The novel kinases and phosphatases of the present invention can be assumed to be closely associated with intracellular physiological functions, and thus, the inventive proteins are useful as target molecules of agents in the development of pharmaceuticals. Furthermore, agents acting on the inventive proteins are expected to be effective pharmaceuticals which can control intracellular physiological functions more precisely than agents represented by previous receptor agonists and antagonists.

What is claimed is:

1. An isolated nucleic acid of any one of (a) to (d) below:
(a) a nucleic acid encoding a protein comprising the amino acid sequence of any one of SEQ ID NO:2, 4, 6, 8, 10, 12, 14, 16, 18, 20, and 22,
(b) a nucleic acid comprising a coding region in the nucleotide sequence of any one of SEQ ID NO:1, 3, 5, 7, 9, 11, 13, 15, 17, 19, or 21,
(c) a nucleic acid encoding a protein that comprises the amino acid sequence of any one of SEQ ID NO:2, 4, 6, 8, 10, 12, 14, 16, 18, 20, and 22, in which one or more amino acids are replaced, deleted, inserted and/or added and that is functionally equivalent to the protein comprising the amino acid sequence of any one of SEQ ID NO:2, 4, 6, 8, 10, 12, 14, 16, 18, 20, and 22, and
(d) a nucleic acid that hybridizes under stringent conditions with the nucleic acid comprising the nucleotide sequence of any one of SEQ ID NOs: 1, 3, 5, 7, 9, 11, 13, 15, 17, 19, or 21, and that encodes a protein finctionally equivalent to the protein comprising the amino acid sequence of any one of SEQ ID NO:2, 4, 6, 8, 10, 12, 14, 16, 18, 20, or 22.

2. An isolated nucleic acid encoding the amino acid sequence of any one of SEQ ID NOs:2, 4, 6, 8, 10, 12, 14, 16, 18, 20, or 22 or a fragment thereof.

3. A vector into which the nucleic acid of claim 1 is inserted.

4. A vector into which the nucleic acid of claim 2 is inserted.

5. A transformant harboring the nucleic acid of claim 1.

6. A transformant harboring the nucleic acid of claim 2.

7. A transformant harboring the vector of claim 3.

8. A transformant harboring the vector of claim 4.

9. A substan t ially purified polypeptide encoded by the nucleic acid of claim 1.

10. A substantially purified polypeptide encoded by the nucleic acid of claim 2.

11. A method for producing a polypeptide, the method comprising the steps of culturing the transformant of claim 7 and recovering a polypeptide expressed from the transformant or the culture supernatant thereof.

12. A method for producing a polypeptide, the method comprising the steps of culturing the transformant of claim 8 and recovering a polypeptide expressed from the transformant or the culture supernatant thereof.

13. An antibody against the polypeptide of claim 9.

14. An antibody against the polypeptide of claim 10.

15. A polynucleotide that hybridizes with the nucleic acid comprising the nucleotide sequence of any one of SEQ ID NO:1, 3, 5, 7, 9, 11, 13, 15, 17, 19, or 21 or the complementary strand thereof and that comprises at least 15 nucleotides.

16. A method for identifying a compound that binds to the polypeptide of claim 9, the method comprising the steps of:
(a) contacting a test sample with the polypeptide or a partial peptide thereof,
(b) detecting a binding activity of the test sample to the polypeptide or the partial peptide thereof, and
(c) selecting a compound comprising the binding activity to the polypeptide or the partial peptide thereof,
thereby identifying a compound that binds to the polypeptide of claim 9.

17. A method for identifying a compound that binds to the polypeptide of claim 10, the method comprising the steps of:
(a) contacting a test sample with the polypeptide or a partial peptide thereof,
(b) detecting a binding activity of the test sample to the polypeptide or the partial peptide thereof, and
(c) selecting a compound comprising the binding activity to the polypeptide or the partial peptide thereof,
thereby identifying a compound that binds to the polypeptide of claim 10.


SEQUENCE LISTING (64 sequences)

SEQ ID NO: 1 - DNA, 2159 bp, Homo sapiens
  CDS (109)...(1713)
cgctgtgagggagtcgctgtgatccggggccccggaacccgagctggagctgaagcgcaggctgcggggcgcggagtcgggagtgcaggcctgagtgttccttccagcatgtcggagggggagtcccagacagtacttagcagtggctcagacccaaaggtagaatcctcatcttcagctcctggcctgacatcagtgtcacctcctgtgacctccacaacctcagctgcttccccagaggaagaagaagaaagtgaagatgagtctgagattttggaagagtcgccctgtgggcgctggcagaagaggcgagaagaggtgaatcaacggaatgtaccaggtattgacagtgcatacctggccatggatacagaggaaggtgtagaggttatgtggaatgaggtacagttctctgaacgcaagaactacaagctgcaggaggaaaaggttcgtgctgtgtttgataatctgattcaattggagcatcttaacattgttaagtttcacaaatattgggctgacattaaagagaacaaggccagggtcatttttatcacagaatacatgtcatctgggagtctgaagcaatttctgaagaagaccaaaaagaaccacaagacgatgaatgaaaaggcatggaagcgttggtgcacacaaatcctctctgccctaagctacctgcactcctgtgacccccccatcatccatgggaacctgacctgtgacaccatcttcatccagcacaacggactcatcaagattggctctgtggctcctgacactatcaacaatcatgtgaagacttgtcgagaagagcagaagaatctacacttctttgcaccagagtatggagaagtcactaatgtgacaacagcagtggacatctactcctttggcatgtgtgcactggagatggcagtgctggagattcagggcaatggagagtcctcatatgtgccacaggaagccatcagcagtgccatccagcttctagaagacccattacagagggagttcattcaaaagtgcctgcagtctgagcctgctcgcagaccaacagccagagaacttctgttccacccagcattgtttgaagtgccctcgctcaaactccttgcggcccactgcattgtgggacaccaacacatgatcccagagaacgctctagaggagatcaccaaaaacatggatactagtgccgtactggctgaaatccctgcaggaccaggaagagaaccagttcagactttgtactctcagtcaccagctctggaattagataaattccttgaagatgtcaggaatgggatctatcctctgacagcctttgggctgcctcggccccagcagccacagcaggaggaggtgacatcacctgtcgtgcccccctctgtcaagactccgacacctgaaccagctgaggtggagactcgcaaggtggtgctgatgcagtgcaacattgagtcggtggaggagggagtcaaacaccacctgacacttctgctgaagttggaggacaaactgagccggcacctgagctgtgacctgatgccaaatgagaatatccccgagttggcggctgagctggtgcagctgggcttcattagtgaggctgaccagagccggttgacttctctgctagaagagaccttgaacaagttcaattttgccaggaacagtaccctcaactcagccgctgtcaccgtctcctcttagagctcactcgggccaggccctgatctgcgctgtggctgtccctggacgtgctgcagccctcctgtcccttccccccagtcagtattaccctgtgaagccccttccctcctttattattcaggagggctgggggggctccctggttctgagcatcatcctttcccctcccctctcttcctcccctctgcactttgtttacttgttttgcacagacgtgggcctgggccttctcagcagccgccttctagttgggggctagtcgctgatctgccggctcccgcccagcctgtgtggaaaggaggcccacgggcactaggggagccgaattctacaatcccgctggggcggccggggcgggagagaaaggtggtgctgcagtggtggccctggggggccattcgattcgcctcagttgctgctgtaataaaagtctactttttgct

SEQ ID NO: 2 - protein, 535 aa, Homo sapiens [sequence omitted]

SEQ ID NO: 3 - DNA, 1818 bp, Homo sapiens
  CDS (170)...(1135)
gaattgcaacggcagctgccgggcgtatgtgttggtgctagaggcagctgcagggtctcgctgggggccaatcgggaccaattttgaggaggtacttggccacgacttattttcacctccgacctttccttccaggcggtgagactctggactgagagtggctttcacaatggaagggatcagtaatttcaagacaccaagcaaattatcagaaaaaaagaaatctgtattatgttcaactccaactataaatatcccggcctctccgtttatgcagaagcttggctttggtactggggtaaatgtgtacctaatgaaaagatctccaagaggtttgtctcattctccttgggctgtaaaaaagattaatcctatatgtaatgatcattatcgaagtgtgtatcaaaagagactaatggatgaagctaagattttgaaaagccttcatcatccaaacattgttggttatcgtgcttttactgaagccagtgatggcagtctgtgtcttgctatggaatatggaggtgaaaagtctctaaatgacttaatagaagaacgatataaagccagccaagatccttttccagcagccataattttaaaagttgctttgaatatggcaagagggttaaagtatctgcaccaagaaaagaaactgcttcatggagacatagagtcttcaaatgttgtaattaaaggcgattttgaaacaattaaaatctgtgatgtaggagtctctctaccactggatgaaaatatgactgtgactgaccctgaggcttgttacattggcacagagccatggaaacccaaagaagctgtggaggagaatggtgttattactgacaaggcagacatatttgcctttggccttactttgtgggaaatgatgactttatcgattccacacattaatctttcaaatgataatgatgatgaagataaaacttttgatgaaagtgattttgatgatgaagcatactatgcagccttgggaactaggccacctattaatatggaagaactggatgaatcataccagaaagtaattgaactcttctctgtatgcactaatgaagaccctaaagatcgtccttctgctgcacacattgttgaagctctggaaacagatgtctagtgatcatctcagctgaagtgtggcttgcgtaaataactgtttattccaaaatatttacatagttactatcagtagttattagactctaaaattggcatatttcaggaccatagtttcttgttaacatatggataactatttctaatatgaaatatgcttatattggctataagcacttggaattgtactgggttttctgtaaagttttagaaactagctacataagtactttgatactgctcatgctgacttaaaacactagcagtaaaacgctgtaaactgtaacattaaattgaatgaccattacttttattaatgatctttcttaaatattctatattttaatggatctactgacattagcactttgtacagtacaaaataaagtctacatttgtttaaaacactgaaccttttgctgatgtgtttatcaaatgataactggaagctgaggagaatatgcctcaaaaagagtagctccttggatacttcagactctggttacagattgtcttgatctcttggatctcctcagatcttctttggtttttgctttaatttattaaatgtattttccatactgagtttaaaatttattaatttgtaccttaagcatttcccagctgtgtaaaaacaataaaactcaaataggatgataaagaataaaggacactttgggt

SEQ ID NO: 4 - protein, 322 aa, Homo sapiens [sequence omitted]

SEQ ID NO: 5 - DNA, 2200 bp, Homo sapiens
  CDS (173)...(1450)
gtcaggccccccagtcttaggtggaaacagcaactccaactcctctggcggggctgggaccgttggtaggggactggtcagtgatggaacgtcccctggggaaagatggactcaccgttttgagaggctgagactcagtcggggagggggcgccttgaaggatggagcagggatggtgcagagggaagagctgctgagtttcatgggggctgaggaggcagcccctgacccagccggagtgggccggggaggaggggtggctgggcctccttcagggggaggagggcagcctcagtggcagaagtgtcgcctgctgcttcgaagtgaaggagaaggaggaggaggaagtcgcctggagttctttgtaccacccaaggcctctcggccccgactcagcatcccctgctcttctatcacagacgtccggacaaccacagccctggagatgcctgaccgggagaacacgtttgtggttaaggtggaaggtccatccgagtatatcatggagacagtggatgcccagcatgtgaaggcctgggtgtctgacatccaagaatgcctgagcccaggaccctgccctgctaccagtccccgccccatgaccctccctctggcccctgggacctcattccttacaagggagaacacagacagcctggagctgtcctgcctgaatcactcggagagtctacccagccaggacctgctgcttggacccagcgagagcaatgaccgcctgtcgcagggggcatatgggggcctctcagaccgcccctcggcatccatctcccccagctctgcctccattgccgcctcccattttgactcgatggaactgcttcccccagagttgcccccccgcatccccattgaagagggacccccagcagggacagttcatcccctctcagccccctaccctcccttggacactccggaaacagccacagggtccttcctgttccagggggagccagagggcggtgagggggaccagcccctctcagggtatccttggttccacgggatgctctctcggctcaaggctgcacagttggtgctgactggcggcactggctcccacggtgtcttcctggtgcgccagagtgagacaaggcggggtgaatacgtcctcaccttcaacttccagggcaaggccaagcacctgcgtttgtcgctgaacgaggagggtcagtgccgggtccagcacctgtggttccagtccattttcgatatgctcgagcacttccgggtgcaccccatccctttggagtcgggaggctccagtgatgttgtccttgtcagctatgtcccatcctcccagcgacagcagggccgggagcaggctgggagccatgcgggggtgtgcgagggagatggatgccaccccgatgcctcctgcaccctcatgcccttcggagcgagtgactgtgtaaccgaccacctcccatgacccaccccagccccctgaacccccttcatggacagatcccccacagcctggggcagaagaggcgtcgagggcgccagaagtggcggcagcagcagccgcagcagccaaagagaggcaagagaaagagaaagcgggcggtggaggggtcccggaagagctggtccccgtggttgagctggtccccgtggttgaattggaagaggccatagccccaggctcagaggcccagggcgctgggtctggtggggacgcgggggtgcccccaatggtgcagctgcagcagtcaccactagggggtgatggagaggaagggggccaccccagggccattaacaaccagtactccttcgtgtgagccaaccccacccgctccaccctttttaaaccccccagccctgctcgtgagattgggctgggtagggacagaggaggccgaaatccctcccccatgcttcctgacccttgttggccaagggcatctttgatggtacaagcagaggctcgggagaggctcccgtcacacactacaggtcccctccccagggcaggggatttgggctccatgagctccttgaggggctcttctggtcagccccaccctgggggccatttccccattaactacccccagcccgaggcagggtgagggggaagggctgtcagttacattaaggtggttgttgttgttgttttaaacaaaatggagaagcataaataaataaaaaggtttatctcggttctatcgtg

SEQ ID NO: 6 - protein, 426 aa, Homo sapiens
MVQREELLSFMGAEEAAPDPAGVGRGGGVAGPPSGGGGQPQWQKCRLLLRSEGEGGGGSRLEFFVPPKASRPRLSIPCSSITDVRTTTALEMPDRENTFVVKVEGPSEYIMETVDAQHVKAWVSDIQECLSPGPCPATSPRPMTLPLAPGTSFLTRENTDSLELSCLNHSESLPSQDLLLGPSESNDRLSQGAYGGLSDRPSASISPSSASIAASHFDSMELLPPELPPRIPIEEGPPAGTVHPLSAPYPPLDTPETATGSFLFQGEPEGGEGDQPLSGYPWFHGMLSRLKAAQLVLTGGTGSHGVFLVRQSETRRGEYVLTFNFQGKAKHLRLSLNEEGQCRVQHLWFQSIFDMLEHFRVHPIPLESGGSSDVVLVSYVPSSQRQQGREQAGSHAGVCEGDGCHPDASCTLMPFGASDCVTDHLP

SEQ ID NO: 7 - DNA, 2720 bp, Homo sapiens
  CDS (3)...(1916)
tcatggaatacgcgagtgggggtgaagtatttgattacttagttgcccatggaagaatgaaagagaaagaggcccgtgcaaaatttaggcagattgtatctgctgtacagtattgtcatcaaaagtacattgttcaccgtgatcttaaggctgaaaaccttctccttgatggtgatatgaatattaaaattgctgactttggttttagtaatgaatttacagttgggaacaaattggacacattttgtggaagcccaccctatgctgctcccgagcttttccaaggaaagaagtatgatggtcctgaagtggatgtgtggagtctgggcgtcattctctatacattagtcagtggctccttgcctttcgatggccagaatttaaaggaactgcgagagcgagttttacgagggaagtaccgtattcccttctatatgtccacagactgtgaaaatcttctgaagaaattattagtcctgaatccaataaagagaggcagcttggaacaaataatgaaagatcgatggatgaatgttggtcatgaagaggaagaactaaagccatatactgagcctgatccggatttcaatgacacaaaaagaatagacattatggtcaccatgggctttgcacgagatgaaataaatgatgccttaataaatcagaagtatgatgaagttatggctacttatattcttctaggtagaaaaccacctgaatttgaaggtggtgaatcgttatccagtggaaacttgtgtcagaggtcccggcccagtagtgacttaaacaacagcactcttcagtcccctgctcacctgaaggtccagagaagtatctcagcaaatcagaagcagcggcgtttcagtgatcatgctggtccatccattcctcctgctgtatcatataccaaaagacctcaggctaacagtgtggaaagtgaacagaaagaggagtgggacaaagatgtggctcgaaaacttggcagcacaacagttggatcaaaaagcgagatgactgcaagccctcttgtagggccagagaggaaaaaatcttcaactattccaagtaacaatgtgtattctggaggtagcatggcaagaaggaatacatatgtctgtgaaaggaccacagatcgatacgtagcattgcagaatggaaaaaacagcagccttacggagatgtctgtgagtagcatatcttctgcaggctcttctgtggcctctgctgccccctcagcacgaccccgccaccagaagtccatgtccacttctggtcatcctattaaagtcacactgccaaccattaaagacggctctgaagcttaccggcctggtacaacccagagagtgcctgctgcttccccatctgctcacagtattagtactgcgactccagaccggacccgttttccccgagggagctcaagccgaagcactttccatggtgaacagctccgggagcgacgcagcgttgcttataatgggccacctgcttcaccatcccatgaaacgggtgcatttgcacatgccagaaggggaacgtcaactggtataataagcaaaatcacatccaaatttgttcgcagaagtacatcaggggaaccaaaagaaagagacaaggaagagggtaaagattctaagccgcgttctttgcggttcacatggagtatgaagaccactagttcaatggaccctaatgacatgatgagagaaatccgaaaagtgttagatgcaaataactgtgattatgagcaaaaagagagatttttgcttttctgtgtccatggagacgctagacaggatagcctcgtgcagtgggagatggaagtctgcaagttgcacgactgtcacttaatggggttcgcttcaagcgaatatctgggacatctattgcctttaagaacattgcatcaaaaatagcaaatgagcttaagctgtaaagaagtccaaatttacaggttcagggaagatacatacatatatgaggtacagtttttgaatgtactggtaatgcctaatgtggtctgcctgtgaatctccccatgtagaatttgcccttaatgcaataaggttatacatagttatgaactgtaaaattaaagtcagtatgaactataataaatatctgtagcttaaaaagtaggttcacatgtacaggtaagtatattgtgtatttctgttcattttctgttcatagagttgtataataaaacatgattgcttaaaaacttgtatagttgtctagatttctgcacctgaatgtatgtttgatgctttgatttgaaaatgttcttccctgttatttacattccggtgggtttttaaaattcttacctccatcatgcaattttgaaaattgtgtccagaattaaaagtgcatagaaatagcctttacaattgtagcatggacctttaaaaattgttttaaaatcttatttaaatttaaaccagaagctgaaaaatagatcagctttattatacacaaaattattactgcttatctttgctcttttccttgttatcccgcaaggtttagttgagaagatacaaaatgtttacagtgttggcacttagagtttttaaattcaagtacatgaaattcagtaatagcattgccttgagctaactaggaagtaccgggaaaaaagttaaatctacatcaagtttcttttgaactttgaagtgttttctgacccactgctaactgtagcaacaaaattt

SEQ ID NO: 8 - protein, 638 aa, Homo sapiens [sequence omitted]

SEQ ID NO: 9 - DNA, 3219 bp, Homo sapiens
  CDS (71)...(2479)
gtagtagctgccaggctgtcccccgccctgcccggcccgagccccgcgggccgccgccgccaccgccgccatgaagaagcagttcaaccgcatgaagcagctggctaaccagaccgtgggcagagctgagaaaacagaagtccttagtgaagatctattacagattgagagacgcctggacacggtgcggtcaatatgccaccattcccataagcgcttggtggcatgtttccagggccagcatggcaccgatgccgagaggagacacaaaaaactgcctctgacagctcttgctcaaaatatgcaagaagcatcgactcagctggaagactctctcctggggaagatgctggagacgtgtggagatgctgagaatcagctggctctcgagctctcccagcacgaagtctttgttgagaaggagatcgtggaccctctgtacggcatagctgaggtggagattcccaacatccagaagcagagggagcagcttgcaagattggtgttagactgggattcagtcagagccaggtggaaccaagctcacaaatcctcaggaaccaactttcaggggcttccatcaaaaatagatactctaaaggaagagatggatgaagctggaaataaagtagaacagtgcaaggatcaacttgcagcagacatgtacaactttatggccaaagaaggggagtatggcaaattctttgttacgttattagaagcccaagcagattaccatagaaaagcattagcagtcttagaaaagaccctccccgaaatgcgagcccatcaagataagtgggcggaaaaaccagcctttgggactcccctagaagaacacctgaagaggagcgggcgcgagattgcgctgcccattgaagcctgtgtcatgctgcttctggagacaggcatgaaggaggagggccttttccgaattggggctggggcctccaagttaaagaagctgaaagctgctttggactgttctacttctcacctggatgagttctattcagacccccatgctgtagcaggtgctttaaaatcctatttacgggaattgcctgaacctttgatgacttttaatctgtatgaagaatggacacaagttgcaagtgtgcaggatcaagacaaaaaacttcaagacttgtggagaacatgtcagaagttgccaccacaaaattttgttaactttagatatttgatcaagttccttgcaaagcttgctcagaccagcgatgtgaataaaatgactcccagcaacattgcgattgtgttagcccctaacttgttatgggccagaaacgaaggaacacttgctgaaatggcagcagccacatccgtccatgtggttgcagtgattgaacccatcattcagcatgccgactggttcttccctgaagaggtggaatttaatgtatcagaagcatttgtacctctcaccaccccgagttctaatcactcattccacactggaaacgactctgactcggggaccctggagaggaagcggcctgctagcatggcggtgatggaaggagacttggtgaagaaggaaagtcctcccaaaccgaaggaccctgtatctgcagctgtgccagcaccagggagaaacaacagtcagatagcatctggccaaaatcagccccaggcagctgctggctcccaccagctctccatgggccaacctcacaatgctgcagggcccagcccgcatacactgcgccgagctgttaaaaaacccgctccagcacccccgaaaccgggcaacccacctcctggccaccccgggggccagagttcttcaggaacatctcagcatccacccagtctgtcaccaaagccacccacccgaagcccctctcctcccacccagcacacgggccagcctccaggccagccctccgccccctcccagctctcagcaccccggaggtactccagcagcttgtctccaatccaagctcccaatcacccaccgccgcagccccctacgcaggccacgccactgatgcacaccaaacccaatagccagggccctcccaaccccatggcattgcccagtgagcatggacttgagcagccatctcacacccctccccagactccaacgccccccagtactccgcccctaggaaaacagaaccccagtctgccagctcctcagaccctggcagggggtaaccctgaaactgcacagccacatgctggaaccttaccgagaccgagaccagtaccaaagccaaggaaccggcccagcgtgcccccacccccccaacctcctggtgtccactcagctggggacagcagcctcaccaacacagcaccaacagcttccaagatagtaacagactccaattccagggtctcagaaccgcatcgcagcatctttcctgaagtgcactcagactcagccagcaaagacgtgcctggccgcatcctgctggatatagacaatgataccgagagcactgccctgtgaagaaagccctttcccagccctccaccacttccaccctggcgagtggagcaggggcaggcgaacctctttctttgcagaccgaacagtgaaaagctttcagtggaggacaaaggagggcctcactgtgtgggacctggccttctgcacggcccaaggagaacctggaggccaccactaaagctgaatgacctgtgtcttgaagaagttggctttctttacatgggaaggaaatcatgccaaaaaaatccaaaacaaagaagtacctggagtggagagagtattcctgctgaaacgcgcataggaagcttttgtccctgctgttaatgcgggcagcacctacagcaacttggaatgagtaagaagcagtgcgttaactatctatttaataaaatgcgctcattatgcaagtcgcctactctctgctacctggacgttcattcttatgtattaggagggaggctgcgctccttcagacttgctgcagaatcattttgtatcatgtatggtctgtgtctccccagtcccctcagaaccatgcccatggatggtgactgctggctctgtcacctcatcaaactggatgtgacccatgccgcctcgttggattgtcggaatgtagacagaaatgtactgttcttttttttttttttaaacaatgtaattgctacttgataaggaccgaacattattctagtttcatgtttaatttgaattaaatatattctgtggtttatatg

SEQ ID NO: 10 - protein, 803 aa, Homo sapiens
MKKQFNRMKQLANQTVGRAEKTEVLSEDLLQIERRLDTVRSICHHSHKRLVACFQGQHGTDAERRHKKLPLTALAQNMQEASTQLEDSLLGKMLETCGDAENQLALELSQHEVFVEKEIVDPLYGIAEVEIPNIQKQREQLARLVLDWDSVRARWNQAHKSSGTNFQGLPSKIDTLKEEMDEAGNKVEQCKDQLAADMYNFMAKEGEYGKFFVTLLEAQADYHRKALAVLEKTLPEMRAHQDKWAEKPAFGTPLEEHLKRSGREIALPIEACVMLLLETGMKEEGLFRIGAGASKLKKLKAALDCSTSHLDEFYSDPHAVAGALKSYLRELPEPLMTFNLYEEWTQVASVQDQDKKLQDLWRTCQKLPPQNFVNFRYLIKFLAKLAQTSDVNKMTPSNIAIVLAPNLLWARNEGTLAEMAAATSVHVVAVIEPIIQHADWFFPEEVEFNVSEAFVPLTTPSSNHSFHTGNDSDSGTLERKRPASMAVMEGDLVKKESPPKPKDPVSAAVPAPGRNNSQIASGQNQPQAAAGSHQLSMGQPHNAAGPSPHTLRRAVKKPAPAPPKPGNPPPGHPGGQSSSGTSQHPPSLSPKPPTRSPSPPTQHTGQPPGQPSAPSQLSAPRRYSSSLSPIQAPNHPPPQPPTQATPLMHTKPNSQGPPNPMALPSEHGLEQPSHTPPQTPTPPSTPPLGKQNPSLPAPQTLAGGNPETAQPHAGTLPRPRPVPKPRNRPSVPPPPQPPGVHSAGDSSLTNTAPTASKIVTDSNSRVSEPHRSIFPEVHSDSASKDVPGRILLDIDNDTESTAL

SEQ ID NO: 11 - DNA, 2224 bp, Homo sapiens
  CDS (215)...(1576)
attctccgggctgcggagggtaaagagcgggctcgggccgaggctggagggctgggtggggccagagcggcgcttcgggggcccgcggaggacgagggagggagagaatctgaggagctgggttgccattaggggactcctgaggtcctatctccaggctgcggtgactgcactttccctggagtggaagctgctggaaggcggaccggccgccatgtccacgttcaggcaggaggacgtggaggaccattatgagatgggggaggagctgggcagcggccagtttgcgatcgtgcggaagtgccggcagaagggcacgggcaaggagtacgcagccaagttcatcaagaagcgccgcctgtcatccagccggcgtggggtgagccgggaggagatcgagcgggaggtgaacatcctgcgggagatccggcaccccaacatcatcaccctgcacgacatcttcgagaacaagacggacgtggtcctcatcctggagctggtctctggcggggagctctttgacttcctggcggagaaggagtcgctgacggaggacgaggccacccagttcctcaagcagatcctggacggcgttcactacctgcactctaagcgcatcgcacactttgacctgaagccggaaaacatcatgctgctggacaagaacgtgcccaacccacgaatcaagctcatcgacttcggcatcgcgcacaagatcgaggcggggaacgagttcaagaacatcttcggcaccccggagtttgtggccccagagattgtgaactatgagccgctgggcctggaggcggacatgtggagcatcggtgtcatcacctatatcctcctgagcggtgcatccccgttcctgggcgagaccaagcaggagacgctcaccaacatctcagccgtgaactacgacttcgacgaggagtacttcagcaacaccggcgagctggccaaggacttcattcgccggctgctcgtcaaagatcccaagcggagaatgaccattgcccagagcctggaacattcctggattaaggcgatccggcggcggaacgtgcgtggtgaggacagcggccgcaagcccgagcggcggcgcctgaagaccacgcgtctgaaggagtacaccatcaagtcgcactccagcctgccgcccaacaacagctacgccgacttcgagcgcttctccaaggtgctggaggaggcggcggccgccgaggagggcctgcgcgagctgcagcgcagccggcggctctgccacgaggacgtggaggcgctggccgccatctacgaggagaaggaggcctggtaccgcgaggagagcgacagcctgggccaggacctgcggaggctacggcaggagctgctcaagaccgaggcgctcaagcggcaggcgcaggaggaggccaagggcgcgctgctggggaccagcggcctcaagcgccgcttcagccgcctggagaaccgctacgaggcgctggccaagcaagtagcctccgagatgcgcttcgtgcaggacctcgtgcgcgccctggagcaggagaagctgcagggcgtggagtgcgggctgcgctaggcgcagtggggtgggccaggccccaggacagccggagctcggcctggggtgggggcgcttcctgtggacgctgcgcctcccatcgcccgggtgcctgtccttgcccagcgccaccaggctggaggcggagtgggaggagctggagccaggcccgtaagttcgcaggcaggggtgggtgtgggacggggctgcttctctacacagcctccacgctggccttcaccttcacccctgcatcgtcggtgaccctgggaccctccaggcagcgtggcctgtggcaccgtgagggttgggacccaccgaggcgcagaggcggcccgaatgcagccctggttcaggcccggaggagggtttgcgggtagttgcacggacaattcggcggggtgctgcctgttgctgccattagcccaggaggaggtcgtgggacggggagggtgggatggacggcggacaggcagtccccacgctgctgggtggcgccgggcttggtggggtcttccactgtgtgcccttctcgccgaggccggtcccccgggtgtggggtgccctgctgcggactcctccgcgagccccatcgtcgcgcctgtggacgcctaggcaagagcggccctctgcagccaagagaaataaaatactggcttccag

SEQ ID NO: 12 - protein, 454 aa, Homo sapiens [sequence omitted]

SEQ ID NO: 13 - DNA, 2778 bp, Homo sapiens
  CDS (773)...(2179)
acaagtggaccggggtgttgggtgctagtcggcaccagaggcaagggtgcgaggaccacggccggctcggacgtgtgaccgcgcctagggggtggcagcgggcagtgcggggcggcaaggcgaccatggagcttttgcggactatcacctaccagccagccgccagcaccaaaatgtgcgagcaggcgctgggcaagggttgcggagcggactcgaagaagaagcggccgccgcagccccccgaggaatcgcagccacctcagtcccaggcgcaagtgcccccggcggcccctcaccaccatcaccaccattcgcactcggggccggagatctcgcggattatcgtcgaccccacgactgggaagcgctactaccggggcaaagtgctgggaaagggtggctttgcaaaatgttacgagatgacagatttgacaaataacaaagtctacgccgcaaaaattattcctcacagcagagtagctaaacctcatcaaagggaaaagattgacaaagaaatagagcttcacagaattcttcatcataagcatgtagtgcagttttaccactacttcgaggacaaagaaaacatttacattctcttggaatactgcagtagaaggtcaatggctcatattttgaaagcaagaaaggtgttgacagagccagaagttcgatactacctcaggcagattgtgtctggactgaataccttcatgaacaagaaatcttgcacagagatctcaaactagggaacttttttattaatgaagccatggaactaaaagttggggacttcggtctggcagccaggctagaacccttggaacacagaaggagaacgatatgtggtaccccaaattatctctctcctgaagtcctcaacaaacaaggacatggctgtgaatcagacatttgggccctgggctgtgtaatgtatacaatgttactagggaggcccccatttgaaactacaaatctcaaagaaacttataggtgcataagggaagcaaggtatacaatgccgtcctcattgctggctcctgccaagcacttaattgctagtatgttgtccaaaaacccagaggatcgtcccagtttggatgacatcattcgacatgacttttttttgcagggcttcactccggacagactgtcttctagctgttgtcatacagttccagatttccacttatcaagcccagctaagaatttctttaagaaagcagctgctgctctttttggtggcaaaaaagacaaagcaagatatattgacacacataatagagtgtctaaagaagatgaagacatctacaagcttaggcatgatttgaaaaagacttcaataactcagcaacccagcaaacacaggacagatgaggagctccagccacctaccaccacagttgccaggtctggaacacccgcagtagaaaacaagcagcagattggggatgctattcggatgatagtcagagggactcttggcagctgtagcagcagcagtgaatgccttgaagacagtaccatgggaagtgttgcagacacagtggcaagggttcttcggggatgtctggaaaacatgccggaagctgattgcattcccaaagagcagctgagcacatcatttcagtgggtcaccaaatgggttgattactctaacaaatatggctttgggtaccagctctcagaccacaccgtcggtgtccttttcaacaatggtgctcacatgagcctccttccagacaaaaaaacagttcactattacgcagagcttggccaatgctcagttttcccagcaacagatgctcctgagcaatttattagtcaagtgacggtgctgaaatacttttctcattacatggaggagaacctcatggatggtggagatctgcctagtgttactgatattcgaagacctcggctctacctccttcagtggctaaaatctgataaggccctaatgatgctctttaatgatggcacctttcaggtgaatttctaccatgatcatacaaaaatcatcatctgtagccaaaatgaagaataccttctcacctacatcaatgaggataggatatctacaactttcaggctgacaactctgctgatgtctggctgttcatcagaattaaaaaatcgaatggaatatgccctgaacatgctcttacaaagatgtaactgaaagacttttcgaatggaccctatgggactcctcttttccactgtgagatctacagggaagccaaaagaatgatctagagtatgttgaagaagatggacatgtggtggtacgaaaacaattcccctgtggcctgctggactggttggaaccagaacaggctaaggcatacagttcttgactttggacaatccaagagtgaaccagaatgcagttttccttgagatacctgttttaaaaggtttttcagacaattttgcagaaaggtgcattgattcttaaattctctctgttgagagcatttcagccagaggactttggaactgtgaatatacttcctgaaggggagggagaagggaggaagctcccatgttgtttaaaggctgtaattggagcagcttttggctgcgtaactgtgaactatggccatatataattttttttcattaatttttgaagatacttgtggctggaaaagtgcattccttgttaataaactttttatttattacagcccaaagagcagtatttattatcaaaatgtctttttttttatgttgaccattttaaaccgttggcaataaagagtatgaaaacgc

SEQ ID NO: 14 - protein, 469 aa, Homo sapiens [sequence omitted]

SEQ ID NO: 15 - DNA, 2574 bp, Homo sapiens
  CDS (23)...(2290)
attcttgcatagcagtgtgaaaatggtgcatggaaatatcactcctgaaaatataattttgaataagagtggagcctggaaaataatgggttttgatttttgtgtatcatcaaccaatccttctgaacaagagcctaaatttccttgtaaagaatgggacccaaatttaccttcattgtgtcttccaaatcctgaatatttggctcctgaatacatactttctgtgagctgtgaaacagccagtgatatgtattctttaggaactgttatgtatgctgtatttaataaagggaaacctatatttgaagtcaacaagcaagatatttacaagagtttcagtaggcagttggatcagttgagtcgtttaggatctagttcacttacaaatatacctgaggaagttcgtgaacatgtaaagctactgttaaatgtaactccgactgtaagaccagacgcagatcaaatgacaaagattcccttctttgatgatgttggtgcagtaacactgcaatattttgataccttattccaaagagataatcttcagaaatcacagtttttcaaaggactgctaaaggttctaccaaaactgcccaagcgtgtcattgtgcagagaattttgccttgtttgacttcagaatttgtaaaccctgacatggtaccttttgttttgcccaatgttctacttattgctgaggaatgcaccaaagaagaatatgtcaaattaattcttcctgaacttggccctgtgtttaagcagcaggagccaatccagattttgttaattttcctacaaaaaatggatttgctactaaccaaaacccctcctgatgagataaagaacagtgttctacccatggtttacagagcactagaagctccttccattcagatccaggagctgtgtctaaacatcattccaacctttgcaaatcttatagactacccatccatgaaaaacgctttgataccaagaattaaaaatgcttgtctacaaacatcttcccttgcggttcgtgtaaattcattagtgtgcttaggaaagattttggaatacttggataagtggtttgtacttgatgatatcctacccttcttacaacaaattccatccaaggaacctgcggtcctcatgggaattttaggtatttacaaatgtacttttactcataagaagttgggaatcaccaaagagcagctggccggaaaagtgttgcctcatcttattcccctgagtattgaaaacaatcttaatcttaatcagttcaattctttcatttccgtcataaaagaaatgcttaatagattggagtctgaacataagactaaactggagcaacttcatataatgcaagaacagcagaaatctttggatataggaaatcaaatgaatgtttctgaggagatgaaagttacaaatattgggaatcagcaaattgacaaagtttttaacaacattggagcagaccttctgactggcagtgagtccgaaaataaagaggacgggttacagaataaacataaaagagcatcacttacacttgaagaaaaacaaaaattagcaaaagaacaagagcaggcacagaagctgaaaagccagcagcctcttaaaccccaagtgcacacacctgttgctactgttaaacagactaaggacttgacagacacactgatggataatatgtcatccttgaccagcctttctgttagtacccctaaatcttctgcttcaagtactttcacttctgttccttccatgggcattggtatgatgttttctacaccaactgataatacaaagagaaatttgacaaatggcctaaatgccaatatgggctttcagacttcaggattcaacatgcccgttaatacaaaccagaacttctacagtagtccaagcacagttggagtgaccaagatgactctgggaacacctcccactttgccaaacttcaatgctttgagtgttcctcctgctggtgcaaagcagacccaacaaagacccacagatatgtctgcccttaataatctctttggccctcagaaacccaaagttagcatgaaccagttatcacaacagaaaccaaatcagtggcttaatcagtttgtacctcctcaaggttctccaactatgggcagttcagtaatggggacacagatgaacgtgataggacaatctgcttttggtatgcagggtaatcctttctttaacccacagaactttgcacagccaccaactactatgaccaatagcagttcagctagcaatgatttaaaagatctttttgggtgaggtgtcttacttctattttgaaggattatttcagtttcaatcatgggtgagctgatttacatctttatatagttggcttggaggaagtactcctatgggaaagtgaacagttctgtgacaggaaacatctctgtccatgccagcatagtagttgtatggacttctaaccagttgagttttttaaagcattgaggattttttcctcttaccaactcctcttcaggtttttaaagccccagcgcctatattaaggcacatttgaataaattctattaccagtt

SEQ ID NO: 16 - protein, 756 aa, Homo sapiens [sequence omitted]

SEQ ID NO: 17 - DNA, 1074 bp, Homo sapiens
  CDS (67)...(690)
gcagtaccagtttgtgcaccacgtcatgagcctctacgaaaagcagctgtcccaccagtccccagaatgactgcgcttctcctacaaggttctctgggcactgcccagcctgagtctcggccctcacccagggccctgcctcgggtcctgggcctgctccccgcttcctccccttcagtcagctccctctgtcctctgtcagcctggcctgacccctaccctccagcattgctcttcctactgtacatattggggagtggggggcagggtcgggaagggacatgccaggccaggcctggggccccggggcctgacccacaccacgcagaccccgggctccagtttttaacgatggttccatcaatacctgatccagaatgtttccgtgctacactttgtgtcctgctgcaatgtgttctgtctgtccatccatctctgccctctgtaccggacactgtgtctcctcagccaggaaggggtaatgagctccagcccctaagcaaccggacttgcctgcctcggcctcacccgcacttctcccaaaaggcagatgacggggagttaggcatggggagctccagaaggtcaccagagagctttcagctgagggagagttctctaggttggagtgggcatcacagccagggtggcctctgggtgtcagatgctctcaggagggtgcccagcctgtgaggcactggcaaggtagggggcagatggggcatggagaacccagaggatctaggccctgttggggaggggaggggagctcaaggtttgggtggggactcagcccagatctacgtgagacatttttctgtgtcactgtgggaaagccttcccagaagtctcactgcgtgttgctctgcgtgtgttcccatgtccgtgcgtgtgttgagagcccatcaggagggcatgcatgactctttggcaacatgtattatcttggagccacgtgtttttattgctgactttaaatatttatcccacggcagacagagacatttggtgtctttttataattcgctcgtggtcattgaatagagcaataaacggagcattttgagcaaaact

SEQ ID NO: 18 - protein, 208 aa, Homo sapiens
MTALLLQGSLGTAQPESRPSPRALPRVLGLLPASSPSVSSLCPLSAWPDPYPPALLFLLYILGSGGQGREGTCQARPGAPGPDPHHADPGLQFLTMVPSIPDPECFRATLCVLLQCVLSVHPSLPSVPDTVSPQPGRGNELQPLSNRTCLPRPHPHFSQKADDGELGMGSSRRSPESFQLRESSLGWSGHHSQGGLWVSDALRRVPSL

SEQ ID NO: 19 - DNA, 1959 bp, Homo sapiens
  CDS (1357)...(1929)
agaaaaaatgtgctgcgttctgaaaaataactccttagcttggtctgattgttttcagaccttaaaatataaacttgtttcacaagctttaatccatgtggatttttttttcttagagaaccacaaaacataaaaggagcaagtcggactgaatacctgtttccatagtgcccacagggtattcctcacattttctccatagaagatgctttttcccaaggctagaacgacctccaccatgatgaatttgctttttaggtcttaattatttcacttctttttagaaacttaggaagaagtggataatcctgaggtcacacaatctgtcctcccagaaatgaacaaaagtcatcaccttttctgcttgctacacaggcaacgattcccccatcagctgcccggaccctttggcctggcttggtgtgcaggcctgtctgtttgcttaaagtcagtgggttctggtgcagggagtgagaagtgggggaagtgaaagggaaagcatccgtgagaaagcggccacggttttccctccttgtgtgcccatggggcaccagctcatggtctttttcagtcatcccagtttgtacagacttagcttctgaactctaagaatgccaaagggaccgacgagactccccatcacagcgagctctgtccttacatgtatttgatgtgcatcagcggaggagaacactggcttggccctgctccgctgagtgtctgtgaaatacctctactttccctcccatatccagaacaaaatgatacttgacatccttccacaaaagtcagcctaaagaagttatggtatcatatgttaaactaagctttcaaaaaccttagtgaaatagcaagtgactgctttcaagcagcagtcgacatgtaaatgaaggtgttcttagaattcgcattttgccagctcagcgcacctccacaacgaatgaaatgctccgtatgatttgcacaaatgacatagacctccccaaaagttaactggctctccttcctcacacagttcatcataacccaaccccccacccccgggtcatgaaaatcacagaacttataaacacattgaaccctagatctcaggcttcctgacctaccgccagtggccccttgctggccaccctatagggtcctccttccctggcagccccccatgtgggagaaatacctgattctcccaatctgcagtgggagagctttgctgaattccatcccaaagtcaaacatgggcaagaggtgaggatttcacttttaccctcaagtccgatttgtctgtgattttaaactaactgtgtatgtattgatgtttggaagattgtttgaattttaaagtgataatagtacttaatgttatccagtattgttcatcgagacctgaagccgagtaacatccacttcaccgatggctacgagatcaaggaggacatcggggtgggctcctactcagtgtgcaagcgatgtgtgcataaagccacagacaccgagtatgccgtgaagatcattgataagagcaagagagacccctcggaagagattgagatcctcctgcggtacggccagcacccgaacatcatcaccctcaaggatgtctatgatgatggcaagtttgtgtacctggtaatggagctgatgcgtggtggggagctcctggaccgcatcctccggcagagatacttctcggagcgcgaagccagtgacgccctgtgcaccatcaccaagaccatggactacctccattcccagggggttgttcatcgagacctgtttagaggattcagctttgtggcctcaagcctgatccaggagccctcacagcaagatctgcacaaagtcccagttcacccaatcctgctttgtaattcatgtttgagatgggtggccactgtacagatatttattacgctttccagactttctgaatagatttttttgaataaacatggttttatg

SEQ ID NO: 20 - protein, 191 aa, Homo sapiens
MLSSIVHRDLKPSNIHFTDGYEIKEDIGVGSYSVCKRCVHKATDTEYAVKIIDKSKRDPSEEIEILLRYGQHPNIITLKDVYDDGKFVYLVMELMRGGELLDRILRQRYFSEREASDALCTITKTMDYLHSQGVVHRDLFRGFSFVASSLIQEPSQQDLHKVPVHPILLCNSCLRWVATVQIFITLSRLSE

SEQ ID NO: 21 - DNA, 4262 bp, Homo sapiens
  CDS (40)...(2415)
aaaagtgctcgggacaaggacatagggctgagagtagccatgggctctggaggagacagcctcctggggggcaggggttccctgcctctgctgctcctgctcatcatgggaggcatggctcaggactccccgccccagatcctagtccacccccaggaccagctgttccagggccctggccctgccaggatgagctgccaagcctcaggccagccacctcccaccatccgctggttgctgaatgggcagcccctgagcatggtgcccccagacccacaccacctcctgcctgatgggacccttctgctgctacagccccctgcccggggacatgcccacgatggccaggccctgtccacagacctgggtgtctacacatgtgaggccagcaaccggcttggcacggcagtcagcagaggcgctcggctgtctgtggctgtcctccgggaggatttccagatccagcctcgagacatggtggctgtggtgggtgagcagtttactctggaatgtgggccgccctggggccacccagagcccacagtctcatggtggaaagatgagaaacccctggccctccagcccggaaggcacacagtgtccggggggtccctgctgatggcaagagcagagaagagtgacgaagggacctacatgtgtgtggccaccaacagcgcaggacacagggagagccgcgcagcccgggtttccatccaggagccccaggactacacggagcctgtggagcttctggctgtgcgaattcagctggaaaatgtgacactgctgaacccggatcctgcagagggccccaagcctagaccggcggtgtggctcagctggaaggtcagtggccctgctgcgcctgcccaatcttacacggccttgttcaggacccagactgccccgggaggccagggagctccgtgggcagaggagctgctggccggctggcagagcgcagagcttggaggcctccactggggccaagactacgagttcaaagtgagaccatcctctggccgggctcgaggccctgacagcaacgtgctgctcctgaggctgccggaaaaagtgcccagtgccccacctcaggaagtgactctaaagcctggcaatggcactgtctttgtgagctgggtcccaccacctgctgaaaaccacaatggcatcatccgtggctaccaggtctggagcctgggcaacacatcactgccaccagccaactggactgtagttggtgagcagacccagctggaaatcgccacccatatgccaggctcctactgcgtgcaagtggctgcagtcactggtgctggagctggggagcccagtagacctgtctgcctccttttagagcaggccatggagcgagccacccaagaacccagtgagcatggtccctggaccctggagcagctgagggctaccttgaagcggcctgaggtcattgccacctgcggtgttgcactctggctgctgcttctgggcaccgccgtgtgtatccaccgccggcgccgagctagggtgcacctgggcccaggtctgtacagatataccagtgaggatgccatcctaaaacacaggatggatcacagtgactcccagtggttggcagacacttggcgttccacctctggctctcgggacctgagcagcagcagcagcctcagcagtcggctgggggcggatgcccgggacccactagactgtcgtcgctccttgctctcctgggactcccgaagccccggcgtgcccctgcttccagacaccagcactttttatggctccctcatcgctgagctgccctccagtaccccagccaggccaagtccccaggtcccagctgtcaggcgcctcccaccccagctggcccagctctccagcccctgttccagctcagacagcctctgcagccgcaggggactctcttctccccgcttgtctctggcccctgcagaggcttggaaggccaaaaagaagcaggagctgccgcatgccaacagttccccactgctccggggcagccactccttggagctccgggcctgtgagttaggaaatagaggttccaagaacctttcccaaagcccaggagctgtgccccaagctctggttgcctggcgggccctgggaccgaaactcctcagctcccaaatgagctggttactcgtcatctccctccagcacccctctttcctcatgaaactcccccaactcagagtcaacagacccagcctccggtggcaccacaggctccctcctccatcctgctgccagcagcccccatccccatccttagcccctgcagtccccctagcccccaggcctcttccctctctggccccagcccagcttccagtcgcctgtccagctcctcactgtcatccctgggggaggatcaagacagcgtgctgacacctgaggaggtagccctgtgcttggaactcagtgagggtgaggagactcccaggaacagcgtctctcccatgccaagggctccttcaccccccaccacctatgggtacatcagcgtcccaacagcctcagagttcacggacatgggcaggactggaggaggggtggggcccaaggggggagtcttgctgtgcccacctcggccctgcctcacccccacccccagcgagggctccttagccaatggttggggctcagcctctgaggacaatgccgccagcgccagagccagccttgtcagctcctccgatggctccttcctcgctgatgctcactttgcccgggccctggcagtggctgtggatagttttggtttcggtctagagcccagggaggcagactgcgtcttcatagatgcctcatcacctccctccccacgggatgagatcttcctgacccccaacctctccctgcccctgtgggagtggaggccagactggttggaagacatggaggtcagccacacccagcggctgggaagggggatgcctccctggccccctgactctcagatctcttcccagagaagtcagctccactgtcgtatgcccaaggctggtgcttctcctgtagattactcctgaaccgtgtccctgagacttcccagacgggaatcagaaccacttctcctgtccacccacaagacctgggctgtggtgtgtgggtcttggcctgtgtttctctgcagctggggtccaccttcccaagcctccagagagttctccctccacgattgtgaaaacaaatgaaaacaaaattagagcaaagctgacctggagccctcagggagcaaaacatcatctccacctgactcctagccactgctttctcctctgtgccatccactcccaccaccaggttgttttggcctgaggagcagccctgcctgctgctcttcccccaccatttggatcacaggaagtggaggagccagaggtgcctttgtggaggacagcagtggctgctgggagagggctgtggaggaaggagcttctcggagccccctctcagccttacctgggcccctcctctagagaagagctcaactctctcccaacctcaccatggaaagaaaataattatgaatgccactgaggcactgaggccctacctcatgccaaacaaagggttcaaggctgggtctagcgaggatgctgaaggaagggaggtatgggaccgtaggtcaaaagcaccatcctcgtactgttgtcactatgagcttaagaaattcgataccataaaatggtaaagacttgagttctgtgagatcattccccggagcaccatttttaggggagcacctggagagatggcaagaatttcctgagttaggcagggatcaggcattcattgacactcagggagtgtcacacatttctgttctgcaattaaagggagaatgaggttcatccaccaaattttaagcagaatataggaagggcaggggtggggagtttcagggtctgctggtcctggcaacttatattaaatccacctcctcaagataagccttagggatatttatggctgagggaagtgggatggtctaaggcatggggaagggtgattggctgtggggaaaatgaagtaataggttcattctgcccaagtgtacttggggttcatggcatttcataaggacatttgtgtagaaaattgttttgctggctgggcacggtggctcacgcctgtaatcccagcactttgggaggcggaggtaggcagatcatgaggtcaggagatcgagaccatcctggctaacatggtgaaaccccgtctctact

SEQ ID NO: 22 - protein, 792 aa, Homo sapiens [sequence omitted]

SEQ ID NO: 23 - DNA, 1605 bp, Homo sapiens
agcattagtttttgttttttatctgacaggtagctatggatattctgagggagaagccaggattaatacacattttttttttaagttgctgaattgtagtggctctcctttctagcatttttgtcactattgagccctcttagtttatgctagacgtgtttttcttattggttgatattttaaattattaaagccatcttctgaataagctttattcgcactttgtacctagtttctccatcagaaggatctattgctataccattgtatacattttctcattggtcttcgggttactttcagagtgtaaagactccttatgccacaaaattaagcttagatttcccccaaatcaaatactataaatcagattccttagtctagccacaattgacatatcttggagtggataaatctttgttgctggcattgttctgtgcatcataacttgtttagtggcatgtcatcactgtcttctactctctagatgccattagtatactcttcacagttaggacaaccaaaagtgtctccagatattgccaaatgtctcctgatgggcaaagtctatcccagttgcgaaccattattgtaaattaaacttggtttcaaatttgagctttattccttagctctgggaacttgggcaagttacttcccttcgagcctcaatgtcctcatttgtaaaatgacattaatacctacttttagctgtgggaattgagtaccatgatttatacaaagcagtttgtatggtgctggttacatgagagttcagatggtaactagttagtaaaaaatctctagtgtgcttgttgattttattttattttagtatttcttaaagatcaaatttaacatcaatcctaaactttatttagctttttctggcgtgtaaactaacatactaagttgtgtgactataattcatttagtgactcatttttagctatttttataacacattgtgctatggggggttttggaacttgctggaagctacatcagaaactgccatagttaattgccatttcaagaatgttgtaaataactcaggtggccgtttaattctcaatgtaaatataattaactagacatctttcctatatttgtgtctcagttttaaagctatttctggatgcttgagtcttaccgtaattgataacaaaaagaggttattgagaatatctatgatttacagagtaagttattctagacctcaagagtgaaatgtaggggaggagacatttgtgtgttaaactaatggaaatgctcatttaatagatattcactgaaagtattagttttggtttattgtagaaaagttgaggttttatggagatttttgtaaaaaatggtttatttcctaaataaatatctctttttcttttttctcccagaaaatgttaatctatgatccagccaaacgaatttctggcaaaatggcactgaatcatccatattttaatgatttggacaatcagattaagaagatgtagctttctgacaaaaagtttccatatgttatgtcaacagatagttgtgtttttattgttaactcttgtctatttttgtcttatatatatttctttgttatcaaacttc

SEQ ID NO: 24 - DNA, 124 bp, Homo sapiens
  CDS (1)...(123)
ataaatatctctttttcttttttctcccagaaaatgttaatctatgatccagccaaacgaatttctggcaaaatggcactgaatcatccatattttaatgatttggacaatcagattaagaaga

SEQ ID NO: 25 - protein, 41 aa, Homo sapiens
INISFSFFSQKMLIYDPAKRISGKMALNHPYFNDLDNQIKK

SEQ ID NO: 26 - RNA, 30 nt, Artificial Sequence
  Artificially Synthesized Sequence
agcaucgagucggccuuguuggccuacugg

SEQ ID NO: 27 - DNA, 42 bp, Artificial Sequence
  Artificially Synthesized Primer Sequence
gcggctgaagacggcctatgtggccttttttttttttttttt

SEQ ID NO: 28 - DNA, 21 bp, Artificial Sequence
  Artificially Synthesized Primer Sequence
agcatcgagtcggccttgttg

SEQ ID NO: 29 - DNA, 21 bp, Artificial Sequence
  Artificially Synthesized Primer Sequence
gcggctgaagacggcctatgt

SEQ ID NO: 30 - protein, 433 aa, Homo sapiens
MEVVDPQQLGMFTEGELMSVGMDTFIHRIDSTEVIYQPRRKRAKLIGKYLMGDLLGEGSYGKVKEVLDSETLCRRAVKILKKKKLRRIPNGEANVKKEIQLLRRLRHKNVIQLVDVLYNEEKQKMYMVMEYCVCGMQEMLDSVPEKRFPVCQAHGYFCQLIDGLEYLHSQGIVHKDIKPGNLLLTTGGTLKISDLGVAEALHPFAADDTCRTSQGSPAFQPPEIANGLDTFSGFKVDIWSAGVTLYNITTGLYPFEGDNIYKLFENIGKGSYAIPGDCGPPLSDLLKGMLEYEPAKRFSIRQIRQHSWFRKKHPPAEAPVPIPPSPDTKDRWRSMTVVPYLEDLHGADEDEDLFDIEDDIIYTQDFTVPGQVPEEEASHNGQRRGLPKAVCMNGTEAAQLSTKSRAEGRAPNPARKACSASSKIRRLSACKQQ

SEQ ID NO: 31 - protein, 396 aa, Homo sapiens
MPRVKAAQAGRQSSAKRHLAEQFAVGEIITDMAKKEWKVGLPIGQGGFGCIYLADMNSSESVGSDAPCVVKVEPSDNGPLFTELKFYQRAAKPEQIQKWIRTRKLKYLGVPKYWGSGLHDKNGKSYRFMIMDRFGSDLQKIYEANAKRFSRKTVLQLSLRILDILEYIHEHEYVHGDIKASNLLLNYKNPDQVYLVDYGLAYRYCPEGVHKEYKEDPKRCHDGTIEFTSIDAHNGVAPSRRGDLEILGYCMIQWLTGHLPWEDNLKDPKYVRDSKIRYRENIASLMDKCFPEKNKPGEIAKYMETVKLLDYTEKPLYENLRDILLQGLKAIGSKDDGKLDLSVVENGGLKAKTITKKRKKEIEESKEPGVEDTEWSNTQTEEAIQTRSRTRKRVQK

SEQ ID NO: 32 - protein, 297 aa, Homo sapiens
MEDYTKIEKIGEGTYGVVYKGRHKTTGQVVAMKKIRLESEEEGVPSTAIREISLLKELRHPNIVSLQDVLMQDSRLYLIFEFLSMDLKKYLDSIPPGQYMDSSLVKSYLYQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKM

SEQ ID NO: 33 - protein, 403 aa, Homo sapiens
MDRSKENCISGPVKATAPVGGPKRVLVTQQFPCQNPLPVNSGQAQRVLCPSNSSQRIPLQAQKLVSSHKPVQNQKQKQLQATSVPHPVSRPLNNTQKSKQPLPSAPENNPEEELASKQKNEESKKRQWALEDFEIGRPLGKGKFGNVYLAREKQSKFILALKVLFKAQLEKAGVEHQLRREVEIQSHLRHPNILRLYGYFHDATRVYLILEYAPLGTVYRELQKLSKFDEQRTATYITELANALSYCHSKRVIHRDIKPENLLLGSAGELKIADFGWSVHAPSSRRTTLCGTLDYLPPEMIEGRMHDEKVDLWSLGVLCYEFLVGKPPFEANTYQETYKRISRVEFTFPDFVTEGARDLISRLLKHNPSQRPMLREVLEHPWITANSSKPSNCQNKESASKQS

SEQ ID NO: 34 - protein, 344 aa, Homo sapiens
MAQKENSYPWPYGRQTAPSGLSTLPQRVLRKEPVTPSALVLMSRSNVQPTAAPGQKVMENSSGTPDILTRHFTIDDFEIGRPLGKGKFGNVYLAREKKSHFIVALKVLFKSQIEKEGVEHQLRREIEIQAHLHHPNILRLYNYFYDRRRIYLILEYAPRGELYKELQKSCTFDEQRTATIMEELADALMYCHGKKVIHRDIKPENLLLGLKGELKIADFGWSVHAPSLRRKTMCGTLDYLPPEMIEGRMHNEKVDLWCIGVLCYELLVGNPPFESASHNETYRRIVKVDLKFPASVPTGAQDLISKLLRHNPSERLPLAQVSAHPWVRANSRRVLPPSALQSVA

SEQ ID NO: 35 - protein, 745 aa, Homo sapiens
MERPPGLRPGAGGPWEMRERLGTGGFGNVCLYQHRELDLKIAIKSCRLELSTKNRERWCHEIQIMKKLNHANVVKACDVPEELNILIHDVPLLAMEYCSGGDLRKLLNKPENCCGLKESQILSLLSDIGSGIRYLHENKIIHRDLKPENIVLQDVGGKIIHKIIDLGYAKDVDQGSLCTSFVGTLQYLAPELFENKPYTATVDYWSFGTMVFECIAGYRPFLHHLQPFTWHEKIKKKDPKCIFACEEMSGEVRFSSHLPQPNSLCSLIVEPMENWLQLMLNWDPQQRGGPVDLTLKQPRCFVLMDHILNLKIVHILNMTSAKIISFLLPPDESLHSLQSRIERETGINTGSQELLSETGISLDPRKPASQCVLDGVRGCDSYMVYLFDKSKTVYEGPFASRSLSDCVNYIVQDSKIQLPIIQLRKVWAEAVHYVSGLKEDYSRLFQGQRAAMLSLLRYNANLTKMKNTLISASQQLKAKLEFFHKSIQLDLERYSEQMTYGISSEKMLKAWKEMEEKAIHYAEVGVIGYLEDQIMSLHAEIMELQKSPYGRRQGDLMESLEQRAIDLYKQLKHRPSDHSYSDSTEMVKIIVHTVQSQDRVLKELFGHLSKLLGCKQKIIDLLPKVEVALSNIKEADNTVMFMQGKRQKEIWHLLKIACTQSSARSLVGSSLEGAVTPQTSAWLPPTSAEHDHSLSCVVTPQDGETSAQMIEENLNCLGHLSTIIHEANEEQGNSMMNLDWSWLTE

SEQ ID NO: 36 - protein, 318 aa, Homo sapiens [sequence omitted]

SEQ ID NO: 37 - protein, 379 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLSNTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPGVLEAP

SEQ ID NO: 38 - protein, 648 aa, Homo sapiens [sequence omitted]

SEQ ID NO: 39 - protein, 480 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQMITITPPDQDDSMECVDSERRPHFPQFSYSASSTA

SEQ ID NO: 40 - protein, 724 aa, Homo sapiens
MSAEGYQYRALYDYKKEREEDIDLHLGDILTVNKGSLVALGFSDGQEARPEEIGWLNGYNETTGERGDFPGTYVEYIGRKKISPPTPKPRPPRPLPVAPGSSKTEADVEQQALTLPDLAEQFAPPDIAPPLLIKLVEAIEKKGLECSTLYRTQSSSNLAELRQLLDCDTPSVDLEMIDVHVLADAFKRYLLDLPNPVIPAAVYSEMISLAPEVQSSEEYIQLLKKLIRSPSIPHQYWLTLQYLLKHFFKLSQTSSKNLLNARVLSEIFSPMLFRFSAASSDNTENLIKVIEILISTEWNERQPAPALPPKPPKPTTVANNGMNNNMSLQNAEWYWGDISREEVNEKLRDTADGTFLVRDASTKMHGDYTLTLRKGGNNKLIKIFHRDGKYGFSDPLTFSSVVELINHYRNESLAQYNPKLDVKLLYPVSKYQQDQVVKEDNIEAVGKKLHEYNTQFQEKSREYDRLYEEYTRTSQEIQMKRTAIEAFNETIKIFEEQCQTQERYSKEYIEKFKREGNEKEIQRIMHNYDKLKSRISEIIDSRRRLEEDLKKQAAEYREIDKRMNSIKPDLIQLRKTRDQYLMWLTQKGVRQKKLNEWLGNENTEDQYSLVEDDEDLPHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYAQQRR

SEQ ID NO: 41 - protein, 3056 aa, Homo sapiens [sequence omitted]

SEQ ID NO: 42 - protein, 450 aa, Homo sapiens
MSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 43 - protein, 1142 aa, Homo sapiens
MAFCAKMRSSKKTEVNLEAPEPGVEVIFYLSDREPLRLGSGEYTAEELCIRAAQACRISPLCHNLFALYDENTKLWYAPNRTITVDDKMSLRLHYRMRFYFTNWHGTNDNEQSVWRHSPKKQKNGYEKKKIPDATPLLDASSLEYLFAQGQYDLVKCLAPIRDPKTEQDGHDIENECLGMAVLAISHYAMMKKMQLPELPKDISYKRYIPETLNKSIRQRNLLTRMRINNVFKDFLKEFNNKTICDSSVSTHDLKVKYLATLETLTKHYGAEIFETSMLLISSENEMNWFHSNDGGNVLYYEVMVTGNLGIQWRHKPNVVSVEKEKNKLKRKKLENKDKKDEEKNKIREEWNNFSFFPEITHIVIKESVVSINKQDNKKMELKLSSHEEALSFVSLVDGYFRLTADAHHYLCTDVAPPLIVHNIQNGCHGPICTEYAINKLRQEGSEEGMYVLRWSCTDFDNILMTVTCFEKSEQVQGAQKQFKNFQIEVQKGRYSLHGSDRSFPSLGDLMSHLKKQILRTDNISFMLKRCCQPKPREISNLLVATKKAQEWQPVYPMSQLSFDRILKKDLVQGEHLGRGTRTHIYSGTLMDYKDDEGTSEEKKIKVILKVLDPSHRDISLAFFEAASMMRQVSHKHIVYLYGVCVRDVENIMVEEFVEGGPLDLFMHRKSDVLTTPWKFKVAKQLASALSYLEDKDLVHGNVCTKNLLLAREGIDSECGPFIKLSDPGIPITVLSRQECIERIPWIAPECVEDSKNLSVAADKWSFGTTLWEICYNGEIPLKDKTLIEKERFYESRCRPVTPSCKELADLMTRCMNYDPNQRPFFRAIMRDINKLEEQNPDIVSRKKNQPTEVDPTHFEKRFLKRIRDLGEGHFGKVELCRYDPEDNTGEQVAVKSLKPESGGNHIADLKKEIEILRNLYHENIVKYKGICTEDGGNGIKLIMEFLPSGSLKEYLPKNKNKINLKQQLKYAVQICKGMDYLGSRQYVHRDLAARNVLVESEHQVKIGDFGLTKAIETDKEYYTVKDDRDSPVFWYAPECLMQSKFYIASDVWSFGVTLHELLTYCDSDSSPMALFLKMIGPTHGQMTVTRLVNTLKEGKRLPCPPNCPDEVYQLMRKCWEFQPSNRTSFQNLIEGFEALLK

SEQ ID NO: 44 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLLIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 45 - protein, 309 aa, Homo sapiens
MDEKVFTKELDQWIEQLNECKQLSESQVKSLCEKAKEILTKESNVQEVRCPVTVCGDVHGQFHDLMELFRIGGKSPDTNYLFMGDYVDRGYYSVETVTLLVALKVRYRERITILRGNHESRQITQVYGFYDECLRKYGNANVWKYFTDLFDYLPLTALVDGQIFCLHGGLSPSIDTLDHIRALDRLQEVPHEGPMCDLLWSDPDDRGGWGISPRGAGYTFGQDISETFNHANGLTLVSRAHQLVMEGYNWCHDRNVVTIFSAPNYCYRCGNQAAIMELDDTLKYSFLQFDPAPRRGEPHVTRRTPDYFL

SEQ ID NO: 46 - protein, 394 aa, Homo sapiens [sequence omitted]

SEQ ID NO: 47 - protein, 185 aa, Homo sapiens
MSGSFELSVQDLNDLLSDGSGCYSLPSQPCNEVTPRIYVGNASVAQDIPKLQKLGITHVLNAAEGRSFMHVNTNANFYKDSGITYLGIKANDTQEFNLSAYFERAADFIDQALAQKNGRVLVHCREGYSRSPTLVIAYLMMRQKMDVKSALSIVRQNREIGPNDGFLAQLCQLNDRLAKEGKLKP

SEQ ID NO: 48 - protein, 657 aa, Homo sapiens
MRRAVCFPALCLLLNLHAAGCFSGNNDHFLAINQKKSGKPVFIYKHSQDIEKSLDIAPQKIYRHSYHSSSEAQVSKRHQIVNSAFPRPAYDPSLNLLAMDGQDLEVENLPIPAANVIVVTLQMDVNKLNITLLRIFRQGVAAALGLLPQQVHINRLIGKKNSIELFVSPINRKTGISDALPSEEVLRSLNINVLHQSLSQFGITEVSPEKNVLQGQHEADKIWSKEGFYAVVIFLSIFVIIVTCLMILYRLKERFQLSLRQDKEKNQEIHLSPITLQPALSEAKTVHSMVQPEQAPKVLNVVVDPQGRGAPEIRATTATSVCPSPFKMKPIGLQERRGSNVSLTLDMSSLGNIEPFVSIPTPREKVAMEYLQSASRILTRSQLRDVVASSHLLQSEFMEIPMNFVDPKEIDIPRHGTKNRYKTILPNPLSRVCLRPKNVTDSLSTYINANYIRGYSGKEKAFIATQGPMINTVDDFWQMVWQEDSPVIVMITKLKEKNEKCVLYWPEKRGIYGKVEVLVISVNECDNYTIRNLVLKQGSHTQHVKHYWYTSWPDHKTPDSAQPLLQLMLDVEEDRLASQGRGPVVVHCSAGIGRTGCFIATSIGCQQLKEEGVVDALSIVCQLRMDRGGMVQTSEQYEFVHHALCLYESRLSAETVQ

SEQ ID NO: 49 - protein, 537 aa, Homo sapiens
ERLLGRPQPIVMEALDEAEGLQDSQREMPPPPPPSPPSDPAQKPPPRGAGSHSLTVRSSLCLFAASQFLLACGVLWFSGYGHMWSQNATNLVSSLLTLLKQLEPTSWLDSGTWGVPGLLLVFLSVGLVLVTTLVWHLLRTPPEPPTPLPPEDRRQSVSRQPSFTYSEWMEEKIEDDFLDLDPVPETPVFDCVMDIKPEADPTSLTVKSMGLQERRGSNVSLTLDMCTPGCNEEGFGYLMSPREESAREYLLSASRVLQAEELHEKALDPFLLQAEFFEIPMNFVVPKEYDIPGRCRKNRYKTILPNPHSRVCLTSPDPDDPLSSYINANYIRGYGGEEKVYIATQGPIVSTVADFWRMVWQEHTPIIVMITNIEEMNEKCTEYWPEEQVAYDGVEITVQKVIHTEDYRLRLISLKSGTEERGLKHYWFTSWPDQKTPDRAPPLLHLVREVEEAAQQEGPHCAPIIVHCSAGIGRTGCFIATSICCQQLRQEGVVDILKTTCQLRQDRGGMIQHCEQYQFVHHVMSLYEKQLSHQSPE

SEQ ID NO: 50 - protein, 403 aa, Homo sapiens [sequence omitted]

SEQ ID NO: 51 - protein, 447 aa, Homo sapiens
MRSSTLQDPRRRDPQDDVYVDITDRLRFAILYSRPKSASNVHYFSIDNELEYENFSEDFGPLNLAMVYRYCCKINKKLKSITMLRKKIVHFTGSDQRKQANAAFLVGCYMVIYLGRTPEAAYRILIFGDTPYIPFRDAAYGSCNFYITLLDCFHAVKKAMQYGFLNFNSFNLDEYEHYEKAENGDLNWIIPDRFIAFCGPHSRARLESGYHQHSPETYIQYFKNHNVTTIIRLNKRMYDAKRFTDAGFDHHDLFFADGSTPTDAIVKRFLDICENAEGAIAVHCKAGLGRTGTLIACYIMKHYRMTAAETIAWVRICRPGLVIGPQQQFLVMKQTSLWLEGDYFRQRLKGQENGQHRAAFSKLLSGVDDISINGVENQDQQEPKPYSDDDEINGVTQGDRSRALKRRRQSKTNDILLPSPLAVLTFTLCSVVIWWIVCDYILPILLF

SEQ ID NO: 52 - protein, 340 aa, Homo sapiens
MLEAPGPSDGCELSNPSASRVSCAGQMLEVQPGLYFGGAAAVAEPDHLREAGITAVLTVDSEEPSFKAGPGVEDLWRLFVPALDKPETDLLSHLDRCVAFIGQARAEGRAVLVHCHAGVSRSVAIITAFLMKTDQLPFEKAYEKLQILKPEAKMNEGFEWQLKLYQAMGYEVDTSSAIYKQYRLQKVTEKYPELQNLPQELFAVDPTTVSQGLKDEVLYKCRKCRRSLFRSSSILDHREGSGPIAFAHKRMTPSSMLTTGRQAQCTSYFIEPVQWMESALLGVMDGQLLCPKCSAKLGSFNWYGEQCSCGRWITPAFQIHKNRVDEMKILPVLGSQTGKI

SEQ ID NO: 53 - protein, 150 aa, Homo sapiens
MGVQPPNFSWVLPGRLAGLALPRLPAHYQFLLDLGVRHLVSLTERGPPHSDSCPGLTLHRLRIPDFCPPAPDQIDRFVQIVDEANARGEAVGVHCALGFGRTGTMLACYLVKERGLAAGDAIAEIRRLRPGPIETYEQEKAVFQFYQRTK

SEQ ID NO: 54 - protein, 322 aa, Homo sapiens
GLMLRRLRKGNLPIRSIIPNHADKERFATRCKAATVLLYDEATAEWQPEPGAPASVLGLLLQKLRDDGCQAYYLQGGFNKFQTEYSEHCETNVDSSSSPSSSPPTSVLGLGGLRISSDCSDGESDRELPSSATESDGSPVPSSQPAFPVQILPYLYLGCAKDSTNLDVLGKYGIKYILNVTPNLPNAFEHGGEFTYKQIPISDHWSQNLSQFFPEAISFIDEARSKKCGVLVHCLAGISRSVTVTVAYLMQKMNLSLNDAYDFVKRKKSNISPNFNFMGQLLDFERTLGLSSPCDNHASSEQLYFSTPTNHNLFPLNTLEST

SEQ ID NO: 55 - protein, 521 aa, Homo sapiens
MSEPKAIDPKLSTTDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDVYNNKAAVLKYENNVMNIRQFNCSPHPYWLPNFMDVFTWSLPFVGEKVTEMLVNVLNICSDDELGSEEDGFDGATAAARKEVIRNKIRAIGKMARVFSVLREESESVLTLKGLTPTGMLPSGVLSGGKQTLQSATVEAIEADEAIKGFSPQHKITSFEEAKGLDRINERMPPRRDAMPSDANLNSINKALTSETNGTDSNGSNSSNIQ

SEQ ID NO: 56 - protein, 1267 aa, Homo sapiens
DLSRSHCHVYLAHLENSFGPSGAREGSLSSQDSRTESASLSQSQVNGFFASHLGDQTWQESQHGSPSPSVISKATEKETFTDSNQSKTKKPGISDVTDYSDRGDSDMDEATYSSSQDHQTPKQESSSSVNTSNKMNFKTFPSSPPRSGDIFEVELAKNDNSLGISVTGGVNTSVRHGGIYVKAVIPQGAAESDGRIHKGDRVLAVNGVSLEGATHKQAVETLRNTGQVVHLLLEKGQSPTSKEHVPVTPQCTLSDQNAQGQGPEKVKKTTQVKDYSFVTEENTFEVKLFKNSSGLGFSFSREDNLIPEQINASIVRVKKLFPGQPAAESGKIDVGDVILKVNGASLKGLSQQEVISALRGTAPEVFLLLCRPPPGVLPEIDTALLTPLQSPAQVLPNSSKDSSQPSCVEQSTSSDENEMSDKSKKQCKSPSRKDSYSDSSGSGEDDLVTAPANISNSTWSSALHQTLSNMVSQAQSHHEAPRVKKIPFVPCFTILRKRPNKPEFEDSNPSPLPPDMAPGQSYQPQSESASSSSMDKYHIHHISEPTRQENWTPLKNDLENHLEDFELEVELLITLIKSEKGSLGFTVTKGNQRIGCYVHDVIQDPAKSDGRLKPGDRLIKVNDTDVTNMTHTDAVNLLRGSKTVRLVIGRVLELPRIPMLPHLLPDITLTCNKEELGFSLCGGHDSLYQVVYISDINPRSVAAIEGNLQLLDVIHYVNGVSTQGMTLEEVNRALDMSLPSLVLKATRNDLPVVPSSKRSAVSAPKSTKGNGSYSVGSCSQPALTPNDSFSTVAGEEINEISYPKGKCSTYQIKGSPNLTLPKESYIQEDDIYDDSQEAEVIQSLLDVVDEESQNLLNENNAAGYSCGPGTLKMNGKLSEERTEDTDCDGSPLPEYFTEATKMNGCEEYCEEKVKSESLIQKPQEKKTDDDEITWGNDELPIERTNHEDSDKDHSFLTNDELAVLPVVKVLPSGKYTGANLKSVIRVLRVARSGIPSKELENLQELKPLDQCLIGQTKENRRKNRYKNILPYDATRVPLGDEGGYINASFIKIPVGKEEFVYIACQGPLPTTVGDFWQMIWEQKSTVIAMMTQEVEGEKIKCQRYWPNILGKTTMVSNRLRLALVRMQQLKGFVVRAMTLEDIQTREVRHISHLNFTAWPDHDTPSQPDDLLTFISYMRHIHRSGPIITHCSAGIGRSGTLICIDVVLGLISQDLDFDISDLVRCMRLQRHGMVQTEDQYIFCYQVILYVLTRLQAEEEQKQQPQLLK

SEQ ID NO: 57 - protein, 551 aa, Homo sapiens
MNESPDPTDLAGVIIELGPNDSPQTSEFKGATEEAPAKESPHTSEFKGAARVSPISESVLARLSKFEVEDAENVASYDSKIKKIVHSIVSSFAFGLFGVFLVLLDVTLILADLIFTDSKLYIPLEYRSISLAIALFFLMDVLLRVFVERRQQYFSDLFNILDTAIIVILLLVDVVYIFFDIKLLRNIPRWTHLLRLLRLIILLRIFHLFHQKRQLEKLIRRRVSENKRRYTRDGFDLDLTYVTERIIAMSFPSSGRQSFYRNPIKEVVRFLDKKHRNHYRVYNLCSERAYDPKHFHNRVVRIMIDDHNVPTLHQMVVFTKEVNEWMAQDLENIVAIHCKGGTDRTGTMVCAFLIASEICSTAKESLYYFGERRTDKTHSEKFQGVETPSQKRYVAYFAQVKHLYNWNLPPRRILFIKHFIIYSIPRYVRDLKIQIEMEKKVVFSTISLGKCSVLDNITTDKILIDVFDGPPLYDDVKVQFFYSNLPTYYDNCSFYFWLHTSFIENNRLYLPKNELDNLHKQKARRIYPSDFAVEILFGEKMTSSDVVAGSD

SEQ ID NO: 58 - protein, 323 aa, Homo sapiens
MADLDKLNIDSIIQRLLEVRGSKPGKNVQLQENEIRGLCLKSREIFLSQPILLELEAPLKICGDIHGQYYDLLRLFEYGGFPPESNYLFLGDYVDRGKQSLETICLLLAYKIKYPENFFLLRGNHECASINRIYGFYDECKRRYNIKLWKTFTDCFNCLPIAAIVDEKIFCCHGGLSPDLQSMEQIRRIMRPTDVPDQGLLCDLLWSDPDKDVLGWGENDRGVSFTFGAEVVAKFLHKHDLDLICRAHQVVEDGYEFFAKRQLVTLFSAPNYCGEFDNAGAMMSVDETLMCSFQILKPAEKKKPNATRPVTPPRGMITKQAKK

SEQ ID NO: 59 - protein, 319 aa, Homo sapiens
DKLNIDSIIQRLLEVRGSKPGKNVQLQENEIRGLCLKSREIFLSQPILLELEAPLKICGDIHGQYYDLLRLFEYGGFPPESNYLFLGDYVDRGKQSLETICLLLAYKIKYPENFFLLRGNHECASINRIYGFYDECKRRYNIKLWKTFTDCFNCLPIAAIVDEKIFCCHGGLSPDLQSMEQIRRIMRPTDVPDQGLLCDLLWSDPDKDVLGWGENDRGVSFTFGAEVVAKFLHKHDLDLICRAHQVVEDGYEFFAKRQLVTLFSAPNYCGEFDNAGAMMSVDETLMCSFQILKPAEKKKPNATRPVTPPRGMITKQAKK

SEQ ID NO: 60 - protein, 309 aa, Homo sapiens; identical to SEQ ID NO: 45

SEQ ID NO: 61 - DNA, 20 bp, Artificial Sequence
  Artificially Synthesized Primer Sequence
tacggaagtgttacttctgc

SEQ ID NO: 62 - DNA, 20 bp, Artificial Sequence
  Artificially Synthesized Primer Sequence
tgtgggaggttttttctcta

SEQ ID NO: 63 - DNA, 17 bp, Artificial Sequence
  Artificially Synthesized Primer Sequence
gttttcccagtcacgac

SEQ ID NO: 64 - DNA, 17 bp, Artificial Sequence
  Artificially Synthesized Primer Sequence
caggaaacagctatgac